(19) United States
(12) Patent Application Publication
(10) Pub. No.: US 2025/0114309 A1
(43) Pub. Date: Apr. 10, 2025

(54) pH RESPONSIVE POLYMERS AND RELATED METHODS OF SYNTHESIZING, FABRICATING, AND DEPLOYING pH RESPONSIVE POLYMERS

(71) Applicant: VOXCELL BIOINNOVATION INC., Victoria (CA)

(72) Inventors: Karolina Valente, Victoria (CA); Alexandre Brolo, Victoria (CA); Afzal Suleman, Victoria (CA)

(73) Assignee: VOXCELL BIOINNOVATION INC., Victoria, BC (CA)

(21) Appl. No.: 18/725,808

(22) PCT Filed: Feb. 14, 2023
(86) PCT No.: PCT/CA2023/050192

§ 371 Date: Jun. 30, 2024

Related U.S. Application Data
(60) Provisional application No. 63/310,416, filed on Feb. 15, 2022.

(51) Int. Cl. A61K 9/16; A61K 33/242; B01J 13/14; C08H 1/06; C08L 89/06

(52) U.S. Cl. CPC A61K 9/1658; A61K 33/242; B01J 13/14; C08H 1/06; C08L 89/06; C08L 2312/00

(57) ABSTRACT

Aspects of pH responsive polymers are described. One exemplary aspect is a pH responsive GelMA polymer comprising a gelatin type A with selectively modified and unmodified functional groups. For example, this polymer may comprise: modified carboxyl groups, modified hydroxyl groups, unmodified carboxyl groups, unmodified hydroxyl groups, and unmodified amine groups; carboxyl and hydroxyl groups that have been methacrylated; or carboxyl, hydroxyl, and amine groups that were not methacrylated, wherein the modified and unmodified functional groups of Polymer A may be selected by controlling a pH level of its synthesizing reaction. Another exemplary aspect is a pH responsive GelMA polymer comprising a gelatin type B with selectively modified amine groups, modified hydroxyl groups, unmodified carboxyl groups, unmodified, hydroxyl groups, and unmodified amine groups. Related apparatus, compositions, methods, polymers, and systems are described.

### BACKGROUND

### Field of Invention

Aspects of the present disclosure relate to pH responsive polymers and related methods of synthesizing, fabricating, and deploying pH responsive polymers. Particular aspects relate to pH responsive polymers for drug delivery, tissue engineering, and related uses.

### Description of Related Art

Hydrogels are three-dimensional networks of polymeric materials (natural or synthetic) capable of absorbing a considerable amount of water and swelling in an aqueous medium, while maintaining physical integrity due to the presence of hydrophilic functional groups (—OH, —COOH, —SO₃H, and CONH₃). Hydrogels have been applied in the tissue engineering and as drug delivery systems due to their biocompatibility and resemblance to a natural tissue.

Gelatin is a natural polymer originated from collagen, derived through a denaturation process. Depending on the denaturation process (acidic or basic treatment), a gelatin type A or a gelatin type B may be obtained. Gelatin methacryloyl (commonly known as “GelMA”) is a semi-synthetic biomaterial prepared from adding methacrylate and methacrylamide groups to gelatin after reaction with methacrylic anhydride or glycidyl methacrylate. The presence of arginine-glycine-aspartic acid (“RGD”) sequences on GelMA due to its gelatin backbone promotes cell attachment and results in a well applicable biomaterial for biomedical applications.

Due to the presence of different functional groups (amine, hydroxyl, and carboxyl), GelMA can be produced with different degrees of substitution. Most GelMA syntheses focus on reactions between methacrylate groups and amino residues, resulting in the decrease of free lysine amino groups. Although GelMA has extensive applications in biomedical engineering, the synthesis of this polymer has never been tuned towards producing a pH responsive GelMA polymer or hydrogel, the potential applications of which are significant.

### SUMMARY

Aspects of pH responsive polymers are described in this disclosure, including different types of pH responsive GelMA polymers. One aspect of this disclosure is a pH responsive GelMA polymer described as a compound of formula (I). According to this aspect, for example, compound of formula (I) may comprise:

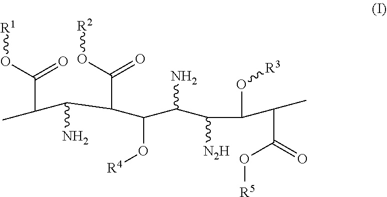

wherein:
the compound comprises a gelatin backbone;
R¹, R², R³, R⁴, and R⁵ are selected from a group consisting of:

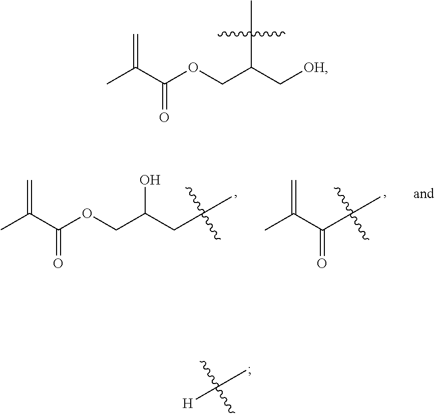

at least one of R¹, R², R³, R⁴, and R⁵ is not

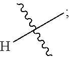 [SMILES: *[H]]

and
“ [symbol] ” indicates the point of attachment.

According to this aspect, to provide additional examples in keeping with this disclosure, the compound of formula (I) also may comprise

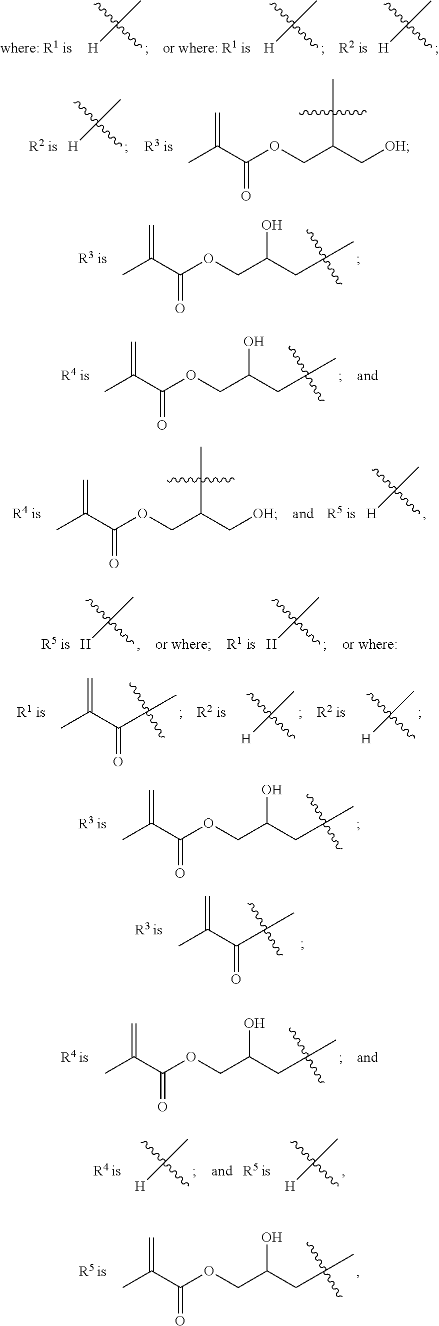

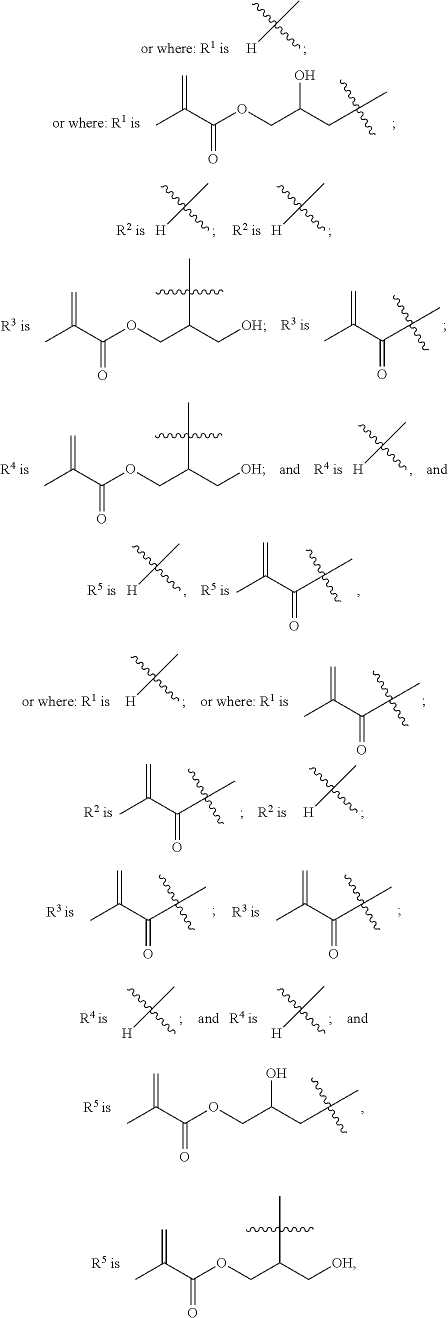

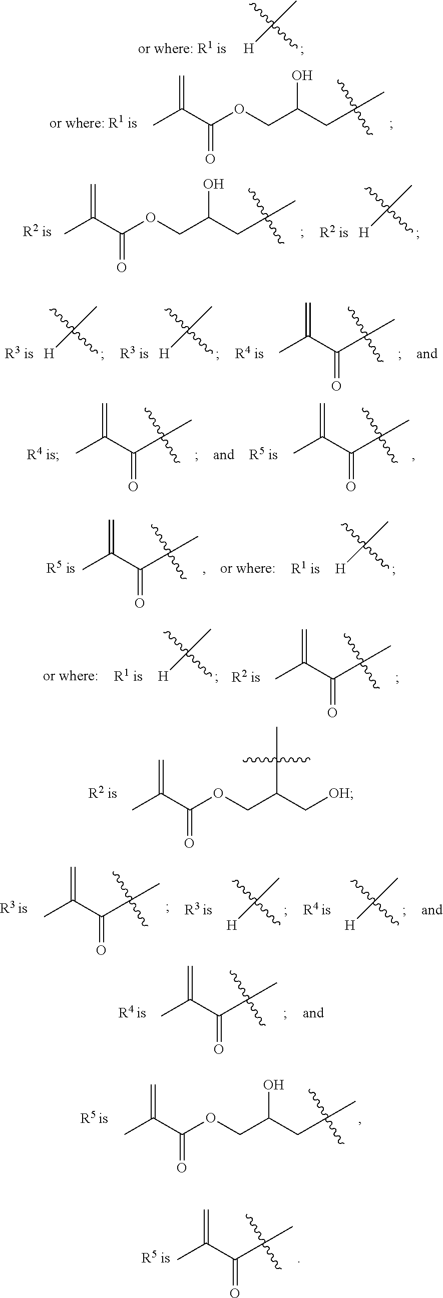

Another aspect of this disclosure is a method of synthesizing a pH responsive polymer, such as a GelMA polymer. According to this aspect, the method may comprise: maintaining a vessel at a target temperature; mixing a gelatin type A in an amount of an acidic water to form a solution in the vessel; stirring the solution while: beginning a reaction by adding glycidyl methacrylate to the solution thereby forming a mixture; conducting the reaction for a first period while: maintaining a target pH of the mixture by measuring a pH of the mixture at intervals during the first period to determine a difference between the measured pH and the target pH; and adding an additional amount of the acidic water to the mixture at each interval of the intervals of the first period so that the measured pH equals the target pH; adding a second amount of the acidic water to the mixture after the first period; conducting the reaction for a second period without maintaining the target pH; adding a third amount of the acidic water to the mixture after the second period; conducting the reaction for a third period; and removing unreacted glycidyl methacrylate from the mixture after the third period.

According to this aspect, maintaining the vessel at the target temperature may comprise placing the vessel in a temperature bath. The method may comprise maintaining the target temperature at approximately 40° C., or at between 40° C. and 50° C. The acidic water may have pH of 3.5. The method may comprise stirring the mixture at a rate of 400 rpm to 500 rpm. The method may comprise adding the glycidyl methacrylate to the solution dropwise at a flowrate, such as between approximately 0.1 mL per minute and approximately 0.5 mL per minute.

The first period may be approximately eighteen hours. The intervals during the first period may occur approximately every five minutes for a first portion of the first period. The first portion of the first period may be approximately three hours. The intervals during the first period may occur approximately every thirty minutes for a second portion of the first period following the first portion of the first period. The second portion of the first period may comprise a remainder of the first period. The intervals of the first period may occur multiple times during each hour of the first period. The target pH of the mixture may be 3.5. The second period may be approximately six hours. The third period may be approximately ten minutes.

Removing the unreacted glycidyl methacrylate from the mixture after the third period may comprise dialyzing the mixture and lyophilizing the mixture. Dialyzing the mixture may comprise transferring the mixture to a dialysis membrane; and performing dialysis, with the dialysis membrane, for a fourth period at a dialysis target temperature. The dialysis target temperature may be 40° C., or approximately 40° C. The fourth period may be approximately seven days, or at least approximately days. The method may comprise adjusting the fourth period relative to a volume of the solution being lyophilized. The method may comprise, prior to lyophilizing the mixture, exposing the mixture to a freezing temperature for a freezing period to create a frozen mixture. The method may comprise placing the frozen mixture in a lyophilizer; and outputting, from the lyophilizer, a dry product of the photoinitiators GelMA polymer. The freezing temperature may be approximately −80° C. The method may comprise freezing the mixture for a freezing period of approximately seven days, or at least three days. The method may comprise adjusting the freezing period relative to a volume of the mixture being lyophilized. The method may comprise synthesizing a hydrogel microsphere or a hydrogel nanosphere. The method may comprise encapsulating molecules or particles in the hydrogel microsphere or the hydrogel nanosphere.

Another aspect of this disclosure is a photoinitiators GelMA polymer comprising a gelatin type A with selectively modified and unmodified functional groups. For example, the polymer may comprise: modified carboxyl groups, modified hydroxyl groups, unmodified carboxyl groups, unmodified hydroxyl groups, and unmodified amine groups; carboxyl and hydroxyl groups that have been methacrylated; or carboxyl, hydroxyl, and amine groups that were not methacrylated, wherein the modified and unmodified functional groups of Polymer A may be selected by controlling a pH level of its synthesizing reaction.

Another aspect of this disclosure is a photoinitiators GelMA polymer comprising: a gelatin type A comprising functional groups comprising modified functional groups and unmodified functional groups, wherein an acidic pH controls which functional groups are modified and unmodified. The modified functional groups may consist of: methacrylated carboxyl groups and methacrylated hydroxyl groups; or methacrylated amine groups and methacrylated hydroxyl groups. The unmodified functional groups may consist of: unreacted amine groups; unreacted carboxyl groups; and unreacted hydroxyl groups. The acidic pH may be 3.5. Additional aspects this disclosure comprise a dry product comprising photoinitiators GelMA polymers like those described herein and a solution comprises such dry products.

Another aspect of this disclosure is a photoinitiators GelMA polymer comprising a gelatin type B with selectively modified amine groups, modified hydroxyl groups, unmodified carboxyl groups, unmodified, hydroxyl groups, and unmodified amine groups. The polymer may comprise: amine and hydroxyl groups that have been methacrylated; and carboxyl, hydroxyl, and amine groups that were not methacrylated.

Another aspect of this disclosure is a method of synthesizing a photoinitiators GelMA polymer comprising: maintaining a vessel at a target temperature of approximately 40° C.; mixing a gelatin type B in an amount of a phosphate buffer to form a solution in the vessel; conducting a reaction for a first period of approximately three hours by stirring the solution in the vessel while adding methacrylic anhydride to the solution thereby forming a mixture; adding additional phosphate buffer solution to the mixture in the vessel and conducting the reaction for a second period of approximately six hours; and removing unreacted methacrylic anhydride from the mixture after the second period by dialyzing the mixture and lyophilizing the mixture.

Another aspect of this disclosure is a method comprising: forming a solution by combining a photoinitiators GelMA polymer with a photoinitiator; flowing the solution through an inner channel of a microfluidic device at a first flow rate while regulating a temperature of the inner channel; flowing a mixture through outer channels of the microfluidic device at a second flow rate; flowing the solution and the mixture into a cross junction of the inner channel and the outer channels, causing a plurality of hydrogel spheres to develop when the solution interacts with the mixture at the cross junction, each hydrogel sphere of the plurality of hydrogel spheres containing a separate volume of the solution; exposing the plurality of hydrogel spheres to an ultraviolet light for a crosslinking period to activate the photoinitiator, creating a plurality of crosslinked hydrogel spheres; and processing the plurality of crosslinked hydrogel spheres for later use.

Forming the solution may comprise combining a dry product of the photoinitiators GelMA polymer with the photoinitiator. The photoinitiators GelMA polymer may comprise Polymer A or Polymer B. The photoinitiator may comprise one or more of: irgacure 2959; lithium phenyl-2,4,6-trimethylbenzoylphosphinate; sodium 3,3′-[(((1E,1′E)-(5-methyl-2-oxocyclohexane-1,3-diylidene)bis(methanylylidene))bis(4,1 phenylene))bis(methylazanediyl)]dipropanoate (E2CK); sodium3,3′-[(((1E,1′E)-(2-oxocyclopentane-1,3-diylidene)bis(methanylylidene))bis(4,1-phenylene))bis(methylazanediyl)]dipropanoate (P2CK); and/or tetrapotassium-4,4′-(1,2-ethenediyl)bis[2-(3-sulfophenyl)diazenesulfonate](AS7). The method may comprise modifying the generally uniform size of the hydrogel spheres by adjusting one or both of the first flow rate and the second flow rate. Regulating the temperature of the inner channel may comprise maintaining the temperature of the inner channel at approximately 40° C.

The solution may comprise: 5% w/v of the photoinitiators GelMA polymer; and 0.5% of the photoinitiator. According to this method, the first flow rate may be 1 μL/min, the second flow rate may be 20 μL/min, and causing a plurality of hydrogel spheres to develop may comprise causing a plurality of hydrogel microspheres to develop, each microsphere of the plurality of microspheres having a generally uniform size. The mixture may comprise a mineral oil and a surfactant; and causing the plurality of hydrogel microspheres to develop may comprise causing the surfactant in the mineral oil to stop the hydrogel microspheres from merging together at the cross junction of the microfluidic device. Exposing the plurality of hydrogel microspheres to the ultraviolet light may comprise: directing the plurality of hydrogel microspheres into a tube; and passing the ultraviolet light through an exterior wall of the tube for a crosslinking period of between approximately 0.5 and approximately 2 hours. Processing the plurality of crosslinked hydrogel microspheres for later use may comprise: exposing the plurality of crosslinked hydrogel microspheres to a dark environment for an exposure time; washing the plurality of crosslinked hydrogel microspheres with Tetrahydrofuran or Hexanes; and storing the plurality of crosslinked hydrogel microspheres in a phosphate buffer solution for a storage time. The exposure time may be overnight, and the storage time may be twenty-four hours.

The solution may comprise: 5% w/v of the photoinitiators GelMA polymer; and between 1 and 0.5% of the photoinitiator. According to this method, the first flow rate may be 4.5 μL/min, the second flow rate may be 400 μL/min, and causing a plurality of hydrogel spheres to develop may comprise causing a plurality of hydrogel nanospheres to develop, each nanosphere of the plurality of nanospheres having a generally uniform size. The mixture may comprise an organic solvent solution and a surfactant; and causing the plurality of hydrogel nanospheres to develop may comprise causing the surfactant in the mixture to stop the hydrogel nanospheres from merging together at the cross junction of the microfluidic device. The organic solvent solution may comprise toluene. The surfactant comprise may span 80™. The method may comprise: collecting the plurality of hydrogel nanospheres in a container; and stabilizing the plurality of hydrogel nanospheres in the container for a stabilization period of approximately twelve hours. Exposing the plurality of hydrogel nanospheres to the ultraviolet light may comprise: directing the plurality of hydrogel nanospheres into a container; and passing the ultraviolet light through an exterior wall of the container for a crosslinking period of between approximately 0.5 and approximately 2 hours.

Processing the plurality of crosslinked hydrogel nanospheres for later use may comprise: exposing the plurality of crosslinked hydrogel nanospheres to a dark environment for an exposure time; washing the plurality of crosslinked hydrogel nanospheres with a solution of Tetrahydrofuran or Hexanes; and storing the plurality of crosslinked hydrogel nanospheres in the solution of Tetrahydrofuran or Hexanes for a storage time. The exposure time may be at least three hours. The storage time is between twenty-four and thirty-six hours.

The photoinitiators GelMA polymer may comprise Polymer A and the method may comprise, after the processing step, one of: exposing the plurality of crosslinked hydrogel spheres to an acidic environment that causes the plurality of crosslinked hydrogel spheres to expand; and exposing the plurality of crosslinked hydrogel spheres to a basic environment that causes the plurality of crosslinked hydrogel spheres to shrink. The photoinitiators GelMA polymer may comprise Polymer A; forming the solution may comprise adding molecules or particles to the solution; and causing the plurality of hydrogel spheres to develop may comprise encapsulating the molecules or particles in the plurality of hydrogel spheres so that each hydrogel sphere of the plurality of hydrogel spheres contains a separate amount the molecules or particles. According to this aspect, the method may comprise releasing the separate amounts of the molecules or particles by exposing the plurality of crosslinked hydrogel spheres to an acidic environment that causes the plurality of crosslinked hydrogel spheres to expand.

The photoinitiators GelMA polymer may comprise Polymer B and the method may comprise, after the processing step, one of: exposing the plurality of crosslinked hydrogel spheres to an acidic environment that causes the plurality of crosslinked hydrogel spheres to shrink; and exposing the plurality of crosslinked hydrogel spheres to a basic environment that causes the plurality of crosslinked hydrogel spheres to expand. The photoinitiators GelMA polymer may comprise Polymer B; forming the solution may comprise adding molecules or particles to the solution; and causing the plurality of hydrogel spheres to develop may comprise encapsulating the molecules or particles in the plurality of hydrogel spheres so that each hydrogel sphere of the plurality of hydrogel spheres contains a separate amount the molecules or particles. According to this aspect, the method may comprise releasing the separate amounts of the molecules or particles by exposing the plurality of crosslinked hydrogel spheres to a basic environment that causes the plurality of crosslinked hydrogel spheres to expand.

The acidic environment may comprise a cancerous environment. The method may comprise adding the molecules or particles to one or both of the photoinitiators GelMA polymer and the photoinitiator. The molecules or particles may comprise a radiotherapy enhancer or sensitizer. The molecules or particles may comprise gold particles.

Another aspect of this disclosure is a method comprising: producing a first plurality of crosslinked hydrogel spheres by combining a first photoinitiators GelMA polymer comprising Polymer A with a first photoinitiator and first molecules or particles to form a first solution, and forming the first plurality of crosslinked hydrogel spheres with the first solution according to method of claim 55; producing a second plurality of crosslinked hydrogel spheres by combining a second photoinitiators GelMA polymer comprising Polymer B with a second photoinitiator and second molecules or particles to form a first solution, and forming the second plurality of crosslinked hydrogel spheres with the second solution according to method of claim 55; and forming a time-released mixture by combining the first plurality of crosslinked hydrogel spheres and the second plurality of crosslinked hydrogel spheres. The method may comprise exposing the time-released mixture to an environment having an initial pH that causes the first plurality of crosslinked hydrogel spheres to swell and burst, releasing the first molecules or particles at a first time; allowing the released first molecules or particles to affect the initial pH at a second time after the first time; and causing the second plurality of crosslinked hydrogel spheres to swell responsive to the affected initial pH until the second molecules or particles are released. The environment may be a cancerous environment, the initial pH may be an acidic pH, and the allowing step may comprise converting the acidic pH into a basic pH. The method may comprise exposing the time-released mixture to an environment having an initial pH that causes the second plurality of crosslinked hydrogel spheres to swell and burst, releasing the second molecules or particles at a first time; allowing the released second molecules or particles to affect the initial pH at a second time after the first time; causing the first plurality of crosslinked hydrogel spheres to swell until the first molecules or particles are released. The environment may be a biological environment, the initial pH may be a basic pH, and the allowing step may comprise converting the basic pH into an acidic pH. The acidic pH may be 5.6 and the basic pH may be 7.4.

Another aspect of this disclosure is method comprising: forming a solution comprising 10% w/v of a photoinitiators GelMA polymer and between 0.045 and 0.1% w/v of a photoinitiator; regulating a temperature of the solution; patterning a tissue construct in the solution by crosslinking select portions of the photoinitiators GelMA polymer with a laser beam configured to the activate the photoinitiator at the select portions; and removing uncrosslinked portions of the photoinitiators GelMA polymer from the container. The photoinitiators GelMA polymer may comprise Polymer A or Polymer B. Regulating the temperature may comprise: depositing the solution in a container; placing the container on a temperature-controlled plate; and regulating the temperature with the temperature-controlled plate by maintaining the temperature of the solution at between approximately 2 and approximately 4° C. The laser beam may be between 300 and 500 nm. The removing step may comprise one or both of: washing the tissue construct with a flow of the solution; and increasing the temperature of the solution to approximately 37° C.

Related apparatus, compositions, methods, polymers, and systems are disclosed.

### BRIEF DESCRIPTION OF THE DRAWINGS

The accompanying drawings, which are incorporated in and constitute part of this disclosure, illustrate exemplary aspects that, together with the written descriptions, explain the principles of this disclosure. Numerous aspects are particularly described, pointed out, and taught in the written descriptions. Some aspects may be even better understood by referencing the written portions together with the accompanying drawings, of which:

FIG. 1 shows an overview of a regular syntheses of a GelMA polymer and two exemplary modified syntheses of pH responsive GelMA polymers, Polymer A and Polymer B.

FIG. 2 shows an exemplary reaction of turning gelatin into Polymer A.

FIG. 3 shows exemplary reaction steps in the synthesis of Polymer A.

FIG. 4 shows an exemplary 1H NMR spectra for Polymer A.

FIG. 5 shows an exemplary reaction of turning gelatin into Polymer B.

FIG. 6 shows exemplary reaction steps in the synthesis for Polymer B.

FIG. 7 shows an exemplary 1H NMR spectra for Polymer B.

FIG. 8 shows an exemplary synthesis reaction of Polymer A.

FIG. 9 shows an exemplary method 300 of synthesizing Polymer A.

FIG. 10 shows an exemplary synthesis reaction of Polymer B.

FIG. 11 shows an exemplary method 400 of synthesizing Polymer B.

FIG. 12 shows an exemplary microfluidic device and indicates a view A-A thereof.

FIG. 13 is a close-up of view A-A of the FIG. 12 microfluidic device.

FIG. 14 is a close-up of view A-A of the FIG. 12 device and indicates a portion B-B.

FIG. 15 shows exemplary hydrogel microspheres of Polymer A after portion B-B of FIG. 14.

FIG. 16 shows exemplary hydrogel microspheres of Polymer B after portion B-B of FIG. 14.

FIG. 17 shows exemplary swelling behaviors of Polymer A hydrogel microspheres under neutral, acidic, and basic mediums; and with red-fluorescent polystyrene nanoparticles.

FIG. 18 shows exemplary swelling behaviors of Polymer B hydrogel microspheres under neutral, acidic, and basic mediums; and with red-fluorescent polystyrene nanoparticles.

FIG. 19 shows exemplary changes in diameter and shrinking/swelling ratios observed for Polymer A in an acidic pH, a neutral pH, and a basic pH.

FIG. 20 shows exemplary changes in diameter and shrinking/swelling ratios observed for Polymer B in an acidic pH, a neutral pH, and a basic pH.

FIG. 21 shows another exemplary microfluidic device and indicates a view C-C thereof.

FIG. 22 a close-up of view C-C of the FIG. 21 microfluidic device.

FIG. 23 a close-up of view C-C of the FIG. 21 device and indicates a portion D-D.

FIG. 24 shows an exemplary system for moving a solution through an inner channel of the FIG. 21 microfluidic device and a mixture through outer channels of the FIG. 21 device.

FIG. 25 shows an exemplary system for activating a photoinitiator in the solution.

FIG. 26 shows exemplary Polymer A or B hydrogel microspheres after portion D-D of FIG. 23.

FIG. 27 shows exemplary Polymer A or B hydrogel microspheres after portion D-D of FIG. 23.

FIG. 28 shows exemplary diameters of Polymer A or B hydrogel microspheres.

FIG. 29 shows exemplary Polymer A or B hydrogel microspheres containing gold particles.

FIG. 30 shows exemplary Polymer A or B hydrogel microspheres containing gold particles.

FIG. 31 shows exemplary diameters of FIGS. 29 and 30 microspheres.

FIG. 32 shows an exemplary a 3D bioprinting system for tissue engineering.

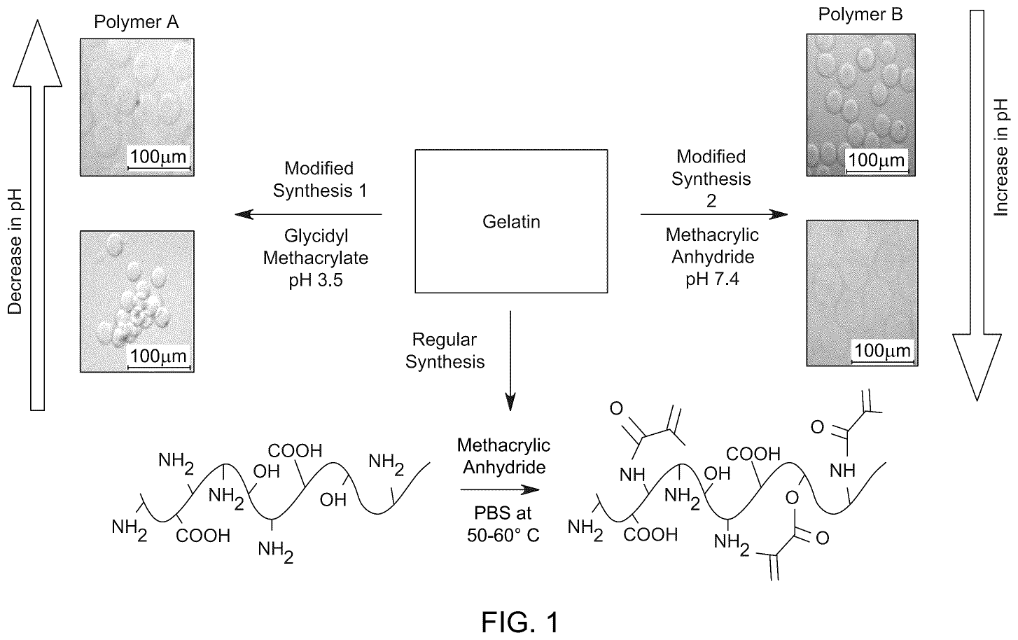

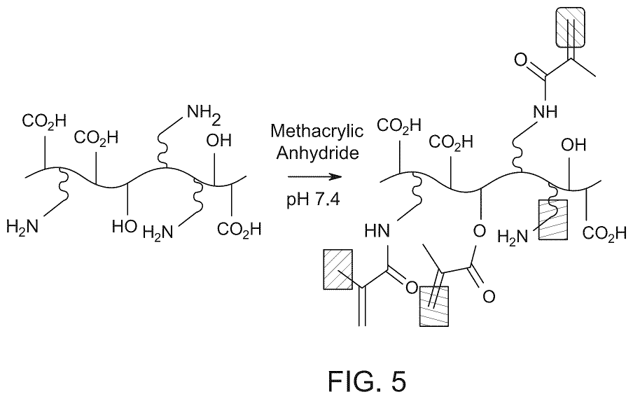

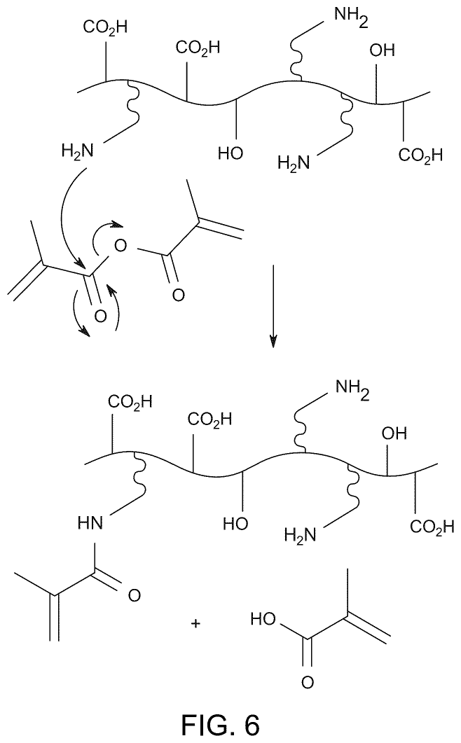

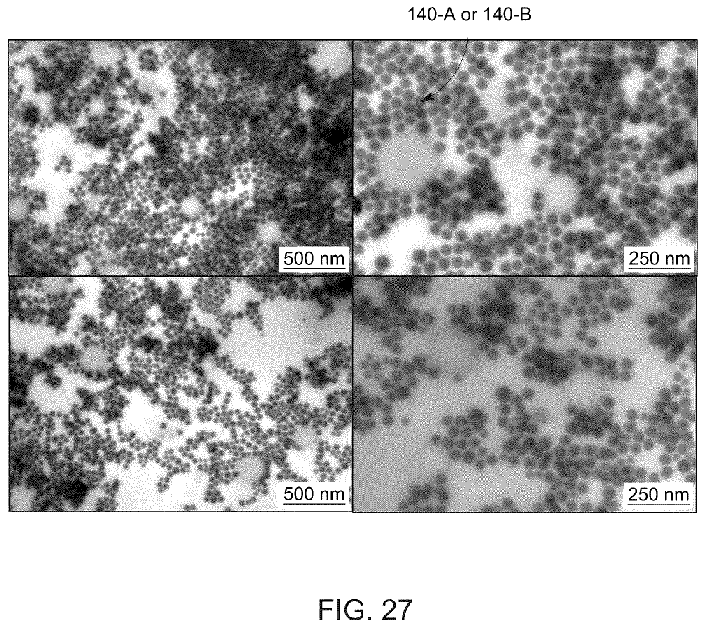

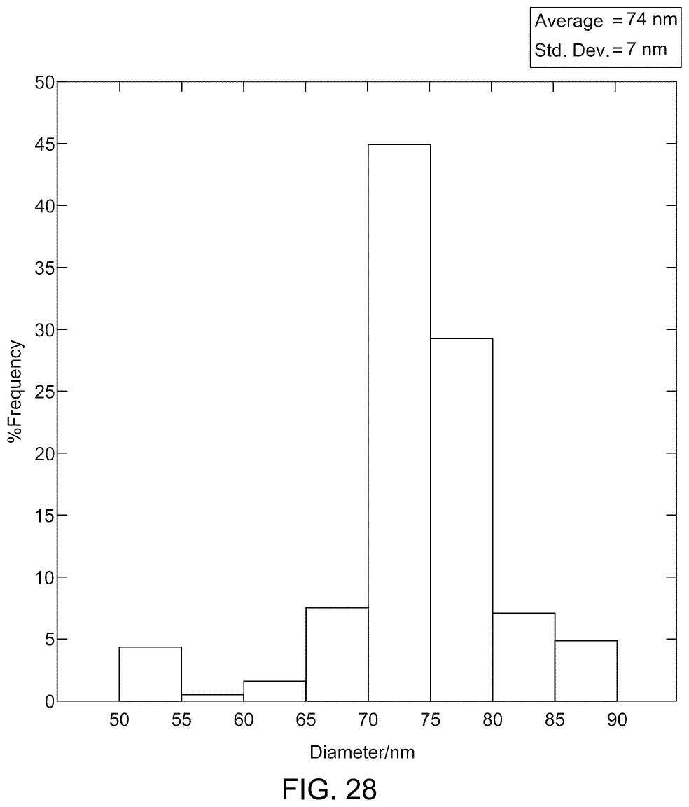

Aspects described or depicted with respect to one or more FIGS. 1 to 32 may be incorporated in different aspects although not specifically described relative thereto. Aspects described herein and/or features thereof may be combined in any way and/or combination. These and other aspects of the present disclosure are explained in detail in the written descriptions contained herein. Further features, advantages, and details of aspects of this disclosure may be appreciated by reading FIGS. 1 to 32 together with the accompanying written descriptions.

### DETAILED DESCRIPTION

Aspects of the present disclosure are not limited to the examples described in the written descriptions and shown in the accompanying drawings. Many aspects of this disclosure may be applicable to other aspects and/or capable of being practiced or carried out in various variants of use, including the examples described herein and shown in the drawings.

Throughout this disclosure, specific details are set forth with particularity in order to provide a more thorough understanding to persons of ordinary skill in the art. For convenience and ease of description, some well-known aspects may be described conceptually to avoid unnecessarily obscuring the focus of this disclosure. In this regard, the written descriptions and accompanying drawings should be interpreted as illustrative rather than restrictive, enabling rather than limiting.

Exemplary aspects of this disclosure reference pH responsive polymers and related methods of synthesizing, fabricating, and deploying pH responsive polymers. Some aspects are described with reference to a particular type of pH responsive polymer (e.g., a pH responsive GelMA polymer like Polymer A), made with a particular method (e.g., one requiring a pH maintaining step), to realize a particular benefit (e.g., predictable swelling characteristics relative to the pH of a medium). Unless claimed, these descriptions are provided for convenience and not intended to limit the present disclosure unless recited in the claims set forth below.

Terms such as “may,” “can,” and like variations, are intended to describe optional aspects of the present disclosure, any of which may be covered by the claims set forth below. Inclusive terms such as “comprises,” “comprising,” “includes,” “including,” and variations thereof, are intended to cover a non-exclusive inclusion, such that an apparatus, method, system, or element thereof comprising a list of elements does not include only those elements but may include other elements not expressly listed and/or inherent thereto.

The term “exemplary” is used in the sense of “example,” rather than “ideal.” The term “and/or” indicates a potential combination, such that a first and/or second element may likewise be described as a first element, a second element, or a combination thereof, each of which being an example. Numerous other combinations are inherent to this disclosure. Various terms of approximation may be used, including “approximately” and “generally.” Approximately means within 10% of and generally means “within most cases” or “usually.”

The term “biocompatible” is intended to describe any material that does not elicit a substantial detrimental response in vivo. The term “R Groups” is intended to describe any combination of R¹, R², R³, R⁴, and R⁵ as those terms are defined below. The term “selectively” is intended to describe intentional change, or not change, by using specific materials or altering the parameters or properties including but not limited to molecules, compounds, polymers, tools, temperature, pH, time, and speed.

The term “solution” is intended to describe any liquid mixture comprising two or more components. Some described solutions may not be truly homogenous and completely free of extraneous materials. The term “mixture” is intended to describe a combination of two more components that may include soluble and/or insoluble ingredients. The term composition is intended to describe an aggregate, mixture, mass, or body formed by combining two or more elements or ingredients. Terms like composition and mixture may be synonymous in that any composition described herein may exist at the moment its ingredients are mixed together.

Aspects of the present disclosure are now described with reference to exemplary polymers, hydrogels, nanogels, compositions; their unique swelling properties; and their potential uses and applications. Some examples may be particularly useful in biological contexts, such as for drug delivery systems and tissue engineering.

Aspects of pH responsive polymers are described. One aspect is a synthesized polymer prepared by controlling the pH of a synthesis reaction with gelatin type A during a specific period, resulting in a pH responsive GelMA polymer that has been selectively modified, described herein as “Polymer A.” Another aspect is a synthesized polymer prepared without controlling the pH of a synthesis reaction with gelatin type B, resulting in a different type of pH responsive GelMA polymer, described herein as “Polymer B.” As described further below, Polymer A may expand and contract oppositely of Polymer B when exposed to different pH mediums.

Exemplary uses for pH responsive GelMA polymers are described. For example, Polymers A and B may be well suited for creating pH responsive hydrogels, drug delivery systems (microgels, nanogels), or bioink compositions with unique and predictable swelling qualities due to selective modifications of the gelatin type A or B contained in corresponding Polymers A or B.

### Polymer A

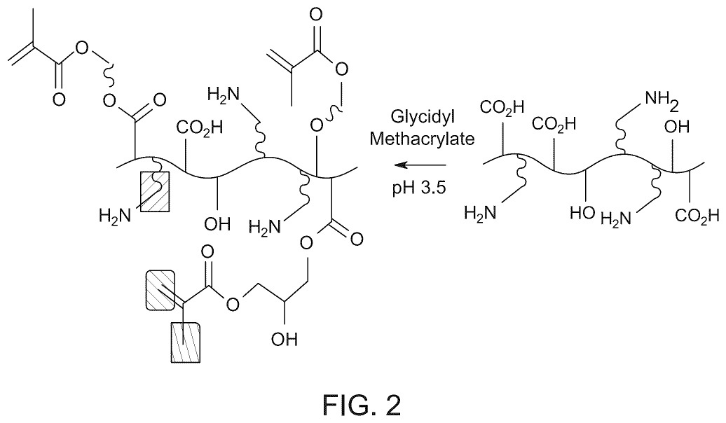

Polymer A may be a pH responsive GelMA polymer comprising a gelatin type A with selectively modified and unmodified functional groups. As shown in FIGS. 1 and 2, Polymer A may comprise a compound of formula (I), each R Group may attach to a carboxyl or hydroxyl group that has been reacted though an epoxide ring-opening mechanism, allowing a methacryl group to attach; each carboxyl or hydroxyl group may not have reacted though an epoxide ring-opening mechanism, allowing the carboxyl or hydroxyl group to remain as its respective functional group; and the amine groups may be reacted though an epoxide ring-opening mechanism, allowing a methacryl group to attach. As shown in FIG. 2, Polymer A may comprise a compound of formula (I) comprising:

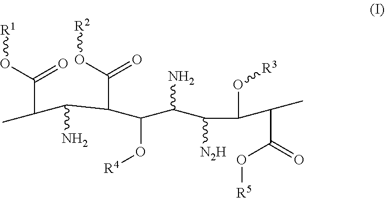

wherein:
the compound comprises a gelatin backbone;
R¹, R², R³, R⁴, and R⁵ are selected from a group consisting of:

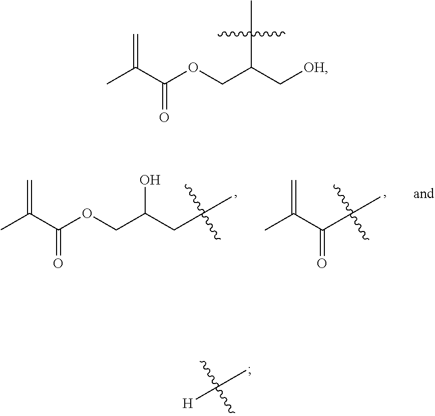

at least one of R¹, R², R³, R⁴, and R⁵ is not

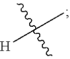 [SMILES: *[H]]

and
“ [symbol] ” indicates the point of attachment.

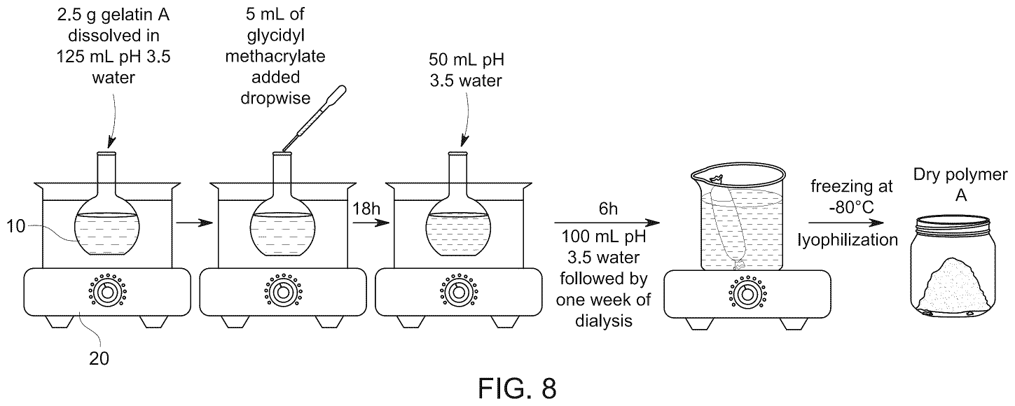

As shown in FIG. 2, Polymer A may comprise a compound of formula (I) comprising: (A) modified carboxyl groups, modified hydroxyl groups, unmodified carboxyl groups, unmodified hydroxyl groups, and unmodified amine groups; (B) carboxyl and hydroxyl groups that have been methacrylated; or (C) carboxyl, hydroxyl, and amine groups that were not methacrylated. As shown in FIG. 8, the modified and unmodified functional groups of Polymer A may be selected by controlling a pH level of its synthesizing reaction.

As shown in FIG. 1, by maintaining pH levels during such reaction: (A) the modified functional groups of Polymer A may consist of (i) methacrylated carboxyl groups and methacrylated hydroxyl groups or (ii) methacrylated amine groups and methacrylated hydroxyl groups; and (B) the unmodified functional groups of Polymer A may consist of (i) unreacted amine groups, (ii) unreacted carboxyl groups, and (iii) unreacted hydroxyl groups.

To provide additional examples for Polymer A, the compound of formula (I) also may comprise

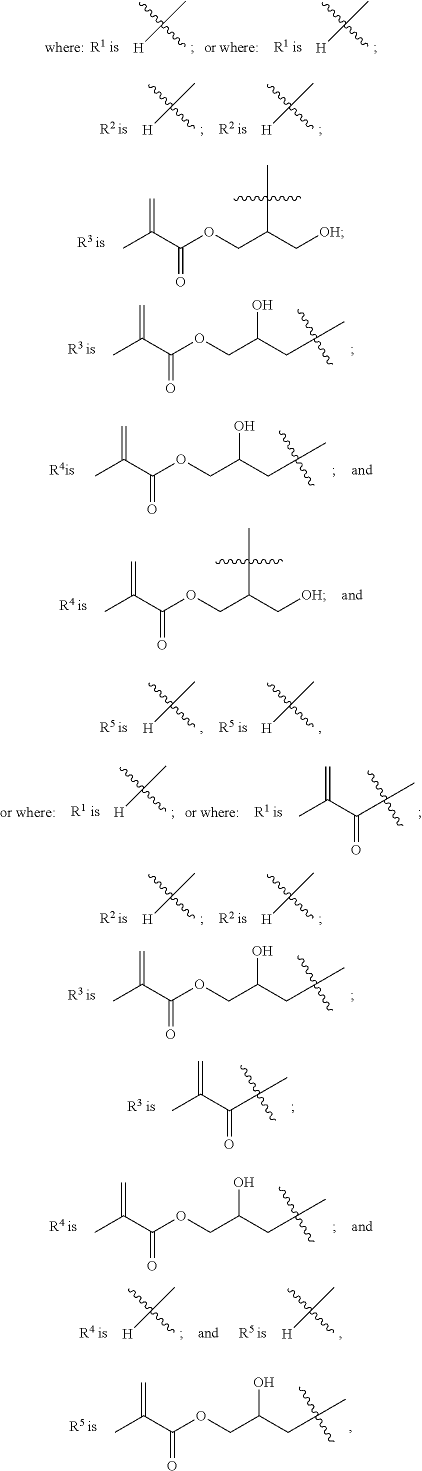

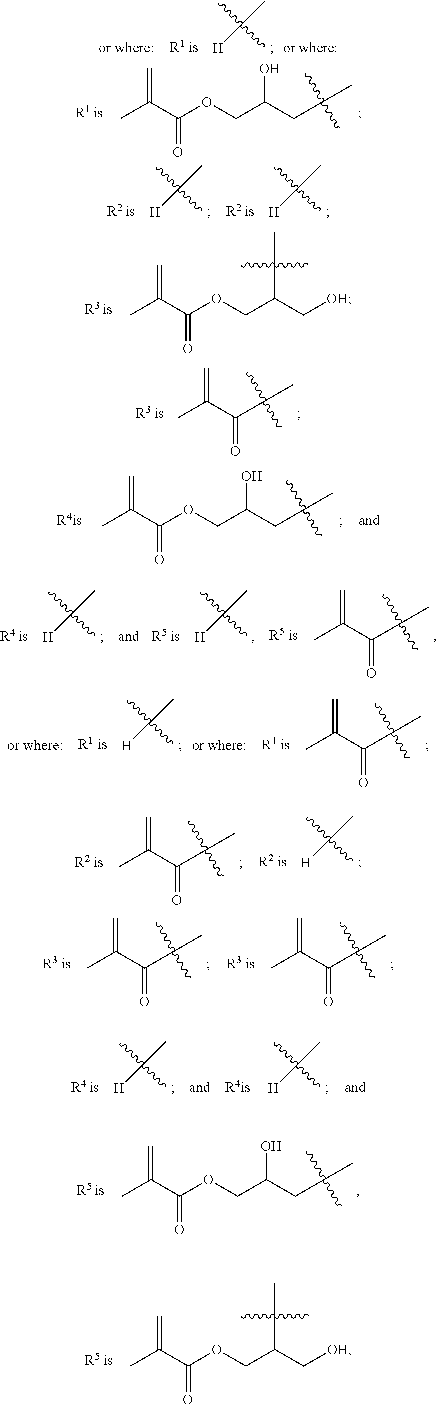

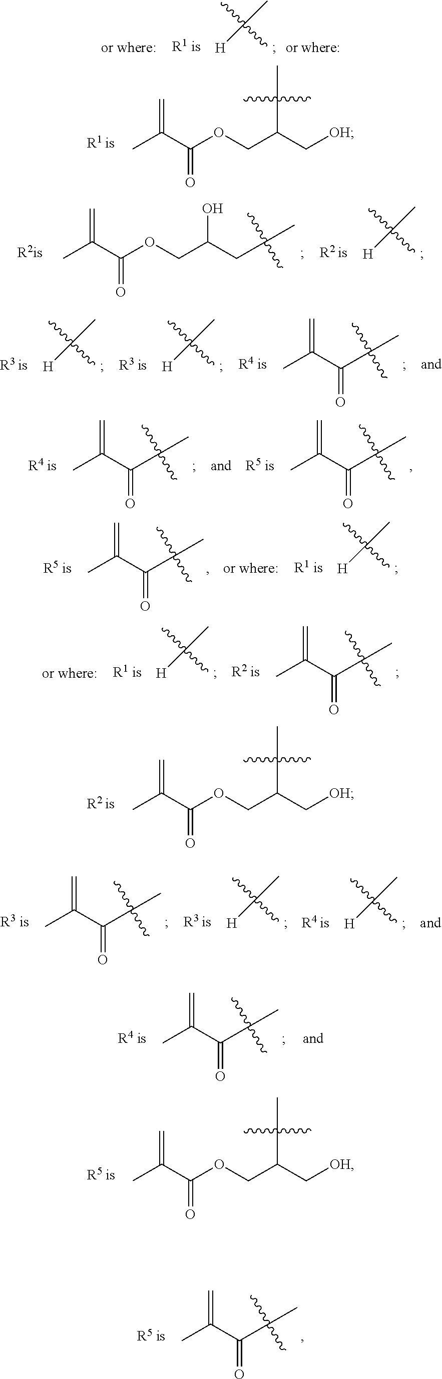

each of the above being examples of Polymer A according to this disclosure.

### Methods of Making Polymer A

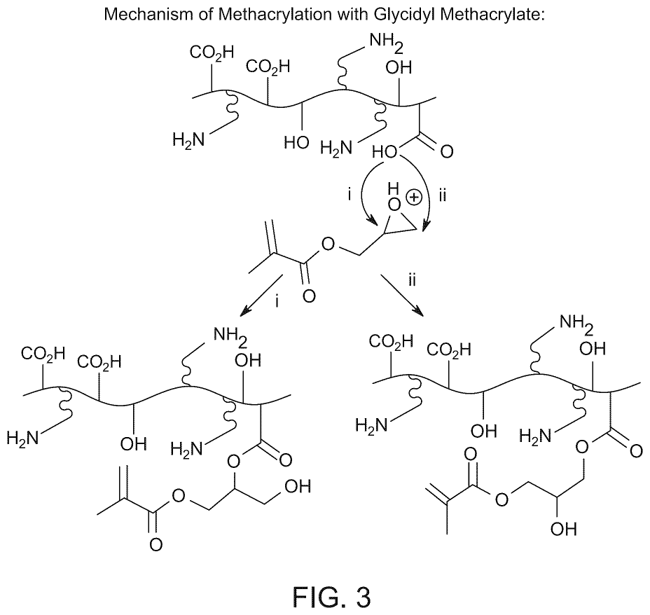

Another aspect of this disclosure is a method 300 of synthesizing a pH responsive GelMA polymer such as Polymer A. Method 300 may comprise modifying a gelatin type A by methacrylating its functional groups. As shown in FIG. 2, method 300 may comprise synthesizing Polymer A by methacrylating the gelatin type A with glycidyl methacrylate, methacrylic anhydride, or an equivalent thereof. As shown in FIG. 3, method 300 may comprise reacting the gelatin type A with glycidyl methacrylate and maintaining an acidic pH of the resulting mixture to achieve the selective modification of its functional groups.

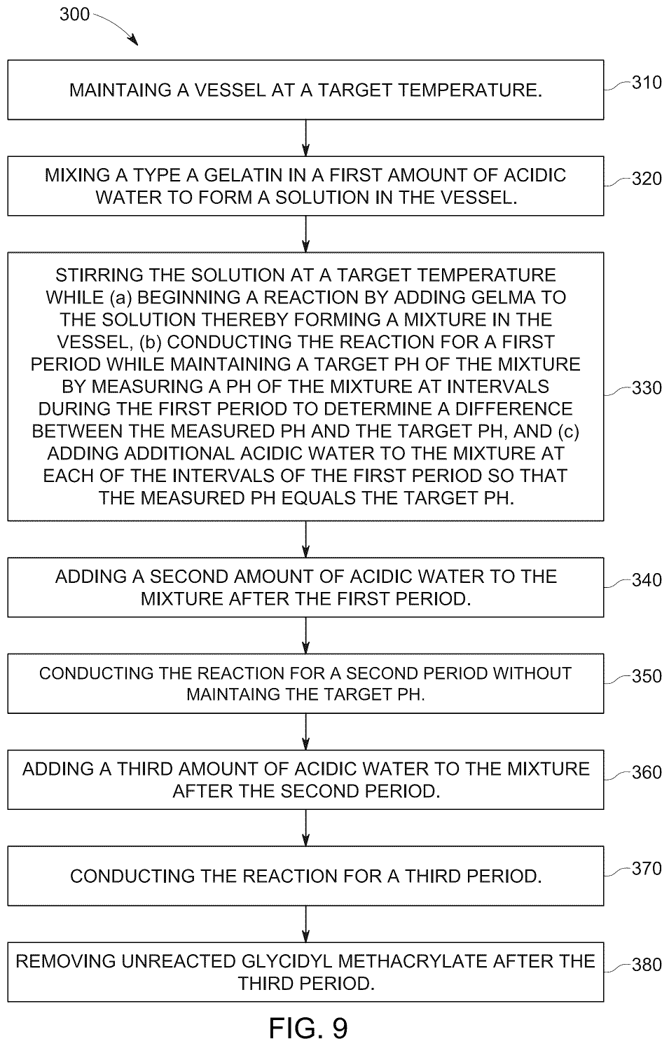

As shown in FIGS. 8 and 9, method 300 may comprise: (i) maintaining a vessel 10 at a target temperature (a maintaining step 310); (ii) mixing a gelatin type A in a first amount of acidic water to form a solution in vessel 10 (a mixing step 320); (iii) stirring the solution at the target temperature while (a) beginning a reaction by adding glycidyl methacrylate to the solution thereby forming a mixture in vessel 10, (b) conducting the reaction for a first period while maintaining a target pH of the mixture by measuring a pH of the mixture at intervals during the first period to determine a difference between the measured pH and the target pH, and (c) adding additional acidic water to the mixture at each of the intervals of the first period so that the measured pH equals the target pH (a modifying step 330); (iv) adding a second amount of acidic water to the mixture after the first period (an additive step 340); (v) conducting the reaction for a second period without maintaining the target pH (a conducting step 350); (vi) adding a third amount of acidic water to the mixture after the second period (an additive step 360); (vii) conducting the reaction for a third period (a conducting step 370); and (xiii) removing unreacted glycidyl methacrylate after the third period (a removal step 380).

As shown in FIGS. 8 and 9, maintaining step 310 may comprise: (a) maintaining a temperature bath 20 at a target temperature; and (b) placing vessel 10 in temperature bath 20. Vessel 10 may comprise a round-bottom flask. Temperature bath 20 may comprise a water bath. The target temperature may be automatically maintained by bath 20 throughout the performance of method 300. For example, the target temperature may be approximately 40° C., or between approximately 40° C. and approximately 50° C., or between 40° C. and 50° C.

As shown in FIGS. 8 and 9, mixing step 320 may comprise forming the solution by adding gelatin type A to vessel 10 and mixing it with reactants in vessel 10. For example, mixing step 320 may comprise forming the solution by: (a) adding 125 mL of acidic water with a pH 3.5 to vessel 10; (b) dissolving 2.5 g of the gelatin type A into the 125 mL of acidic water in vessel 10; and (c) stirring the contents of vessel 10 at approximately 400 rpm, or between 400 rpm and 500 rpm.

While vessel 10 and temperature bath 20 are maintaining the target temperature, modifying step 330 may comprise stirring the solution in vessel 10 at step 320, adding glycidyl methacrylate to form a mixture in vessel 10, and incrementally measuring and adjusting a target pH of the mixture for a first reaction period to selectively modify the gelatin type A. As shown in FIGS. 8 and 9, modifying step 330 may comprise, after forming the step 320 solution, adding 5 mL of glycidyl methacrylate dropwise to the solution at a flow rate, forming the step 330 mixture. The flow rate may be 0.5 mL/min, or between approximately 0.1 m/min and approximately rate 0.5 mL/min. As shown in FIG. 9, the first reaction period including modifying step 330 may be conducted for approximately eighteen hours and comprise manually or automatically readjusting a pH of the step 330 mixture to match a target pH of 3.5 by adding additional amounts of acidic water with a pH 3.5 to vessel 10.

As shown in FIGS. 8 and 9, modifying step 330 may comprise separate readjusting portions during the first reaction period, including a first readjusting portion and a second readjusting portion. The first readjusting portion may comprise approximately three hours of the first reaction period during which modifying step 330 may comprise: (a) manually or automatically measuring a pH of the mixture in vessel 10 at first intervals; and (b) adjusting the pH of the mixture as needed to match the target pH of 3.5 by manually or automatically adding acidic water with a pH 3.5 to the mixture in vessel 10 at each first interval. For example, the first intervals may comprise every five minutes. The second readjusting portion may comprise a remainder of the first reaction period (e.g., approximately fifteen hours) during which step 330 may comprise: (c) manually or automatically measuring the pH of the mixture in vessel 10 at second intervals; and (d) further adjusting the pH as needed to match the target pH (e.g., of 3.5) by manually or automatically adding acidic water with a pH 3.5 to the mixture in vessel 10 at each second interval. For example, the second intervals may comprise every thirty minutes. The first readjusting steps 330 (a) and 330 (b) may be performed early in the first reaction period, before second readjusting steps 330 (c) and 330 (d).

As shown in FIG. 8, additive step 340 may be performed after the first reaction period and comprise adding 50 mL of acidic water with a pH 3.5 to the mixture in vessel 10; and conducting step 350 may comprise allowing the reaction to continue for a second reaction period following the first reaction period. As shown in FIG. 9, the second reaction period may be approximately six hours after the first reaction period.

As shown in FIG. 9, additive step 360 may be performed after the second reaction period and comprise adding 100 mL of acidic water with a pH 3.5 to the mixture in vessel 10; and conducting step 370 may comprise allowing the step 360 mixture to stir for a third reaction period following the second reaction period. As shown in FIGS. 8 and 9, the third period may be approximately ten minutes after the second reaction period.

As shown in FIG. 9, removal step 380 may comprise removing the unreacted glycidyl methacrylate, or equivalent, from the mixture in vessel 10 at step 370. As shown in FIG. 8, step 380 may comprise: (a) transferring the step 370 mixture to dialysis membranes; and (b) removing unreacted glycidyl methacrylate from the mixture with the dialysis membranes. Dialysis steps 380 (a) and (b) may be performed at a target dialysis temperature that may be equal to the target temperature of maintaining step 310, such as 40° C.; and step 380 of method 300 may further comprise: (c) freezing the step 380 (b) mixture to a target freezing temperature of −80° C. to create a frozen mixture; and (d) lyophilizing the step 380 (c) frozen mixture to obtain a dry product of the pH responsive polymer. Method 300 may comprise performing dialysis steps 380 (a) and (d) for a third period of three days and performing freezing step 380 (c) for a fourth period of between at least three days and approximately seven days. Method 300 may comprise adjusting the freezing period relative to a volume of the step 380 mixture being lyophilized. Lyophilizing step 380(d) may comprise placing the frozen solution in a lypholizer; and outputting, from the lyophilizer, the dry product of the pH responsive polymer.

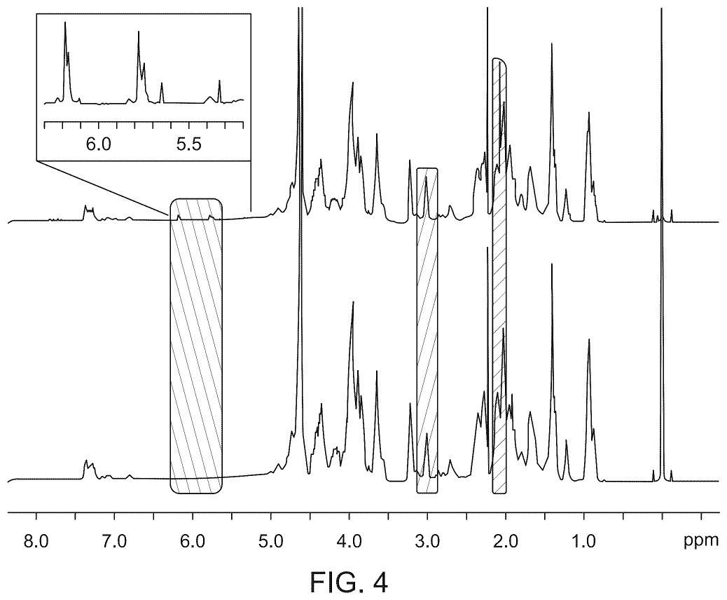

Method 300 may reliably produce pH responsive polymers like Polymer A, which may comprise two pH responsive GelMA fragments, including 3-methacryloyl-1-glycerylester or “GMA1” and 3-methacryloyl-2-glycerylester or “GMA2.” As shown in FIG. 4, the formation of GMA1 and GMA2 fragments in Polymer A may be verified by 1H NMR. The overlapping signals observed at 6.18 ppm and 5.76 ppm correspond to germinal vinyl hydrogens of GMA1 and GMA2. The additional peak at 2.07 ppm seen in the 1H NMR spectrum of Polymer A is associated to the methyl carbon-linked hydrogens at the vinyl carbon, also indicating a reaction of the polymer backbone with glycidyl methylacrylate.

### Polymer B

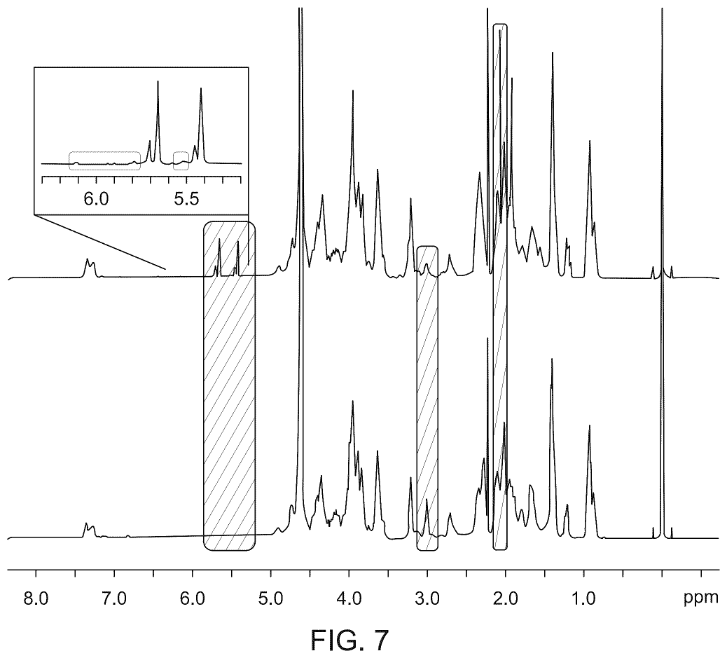

Polymer B may be a pH responsive GelMA polymer comprising a gelatin type B with selectively modified amine groups, modified hydroxyl groups, unmodified carboxyl groups, unmodified, hydroxyl groups, and unmodified amine groups. As shown in FIG. 7, polymer B may comprise a compound with amine and hydroxyl groups that have been methacrylated; and carboxyl, hydroxyl, and amine groups that were not methacrylated. As shown in FIGS. 3A and/or 3B, the gelatin type B may be reacted with methacrylic anhydride using a mechanism of methacrylation.

### Methods of Making Polymer B

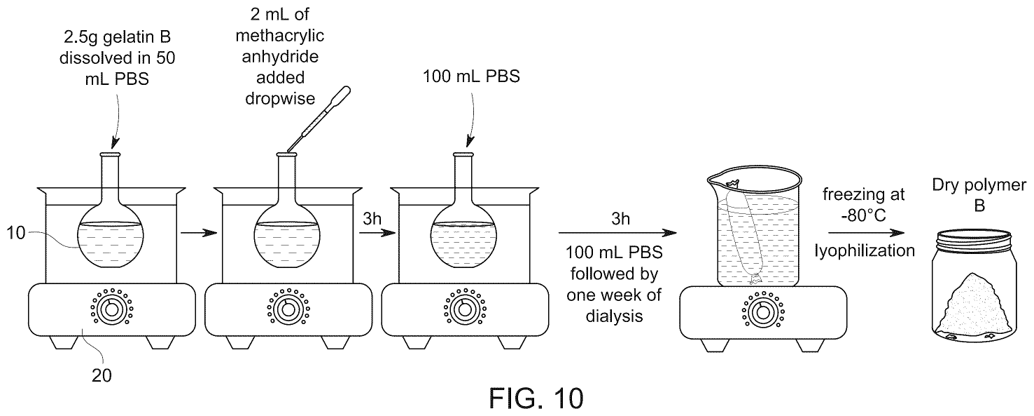

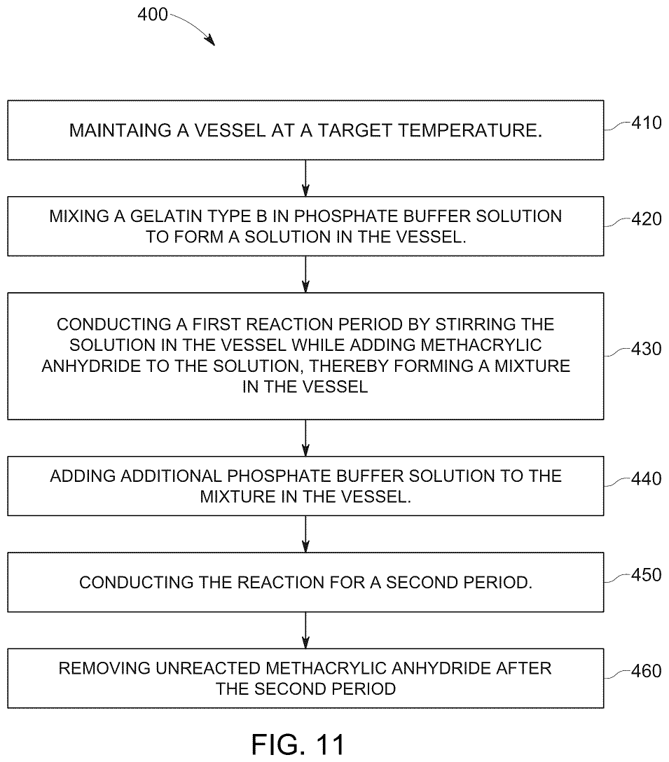

Another aspect of this disclosure is a method 400 of synthesizing a pH responsive polymer from a gelatin type B. Aspects of method 400 are described with reference to a Polymer B derived from gelatin type B. As shown in FIGS. 10 and 11, method 400 may comprise: (i) maintaining vessel 10 at a target temperature (a maintaining step 410); (ii) mixing a gelatin type B in phosphate buffer solution to form a solution in vessel 10 (a mixing step 420); (iii) conducting a first reaction period by stirring the solution in vessel 10 while adding methacrylic anhydride to the solution, thereby forming a mixture in vessel 10 (a modifying step 430); (iv) adding additional phosphate buffer solution to the mixture in vessel 10 (an additive step 440); (v) conducting the reaction for a second period (a conducting step 450); and (xiii) removing unreacted methacrylic anhydride after a third period (a removal step 460).

As shown in FIGS. 10 and 11, maintaining step 410 may comprise: (a) maintaining a temperature bath 20 at a target temperature; and (b) placing a vessel 10 in temperature bath 20. Vessel 10 may comprise a round-bottom flask, as before. Temperature bath 20 may comprise a water bath. The target temperature of water bath 20 may be automatically maintained by bath 20 throughout the performance of method 300. The target temperature may be approximately 40° C., or between approximately 40° C. and approximately 50° C., or between 40° C. and 50° C.

As shown in FIGS. 10 and 11, mixing step 420 may comprise forming the solution by adding the gelatin type B to vessel 10 and mixing it with reactants in vessel 10. For example, mixing step 420 may comprise forming the solution by: (a) adding 50 mL of phosphate buffer solution with a pH 7.4 to vessel 10; (b) dissolving 2.5 g of gelatin type B into the 50 mL of phosphate buffer solution in vessel 10; and (c) stirring the contents of vessel 10 at 400 rpm.

While vessel 10 is maintained at the target temperature, modifying step 430 may comprise stirring the step 420 solution while adding methacrylic anhydride to form a mixture in vessel 10 for a first reaction period to selectively modify the gelatin type B. As shown in FIGS. 10 and 11, modifying step 430 may comprise adding 2 mL of methacrylic anhydride dropwise (rate 0.5 mL/min) to the solution in vessel 10, forming the step 430 mixture. As shown in FIG. 10, the first reaction period including modifying step 430 may be conducted for approximately three hours.

As shown in FIG. 10, additive step 440 may be performed after the first reaction period and comprise adding 100 mL of phosphate buffer solution with a pH 7.4 to the mixture in vessel 10; and conducting step 450 may comprise allowing the reaction to continue for a second reaction period following the first reaction period. As shown in FIG. 10, the second reaction period may be approximately six hours after the first reaction period.

As shown in FIG. 10, removal step 460 may comprise removing the unreacted methacrylic anhydride from the mixture in vessel 10 at step 450. As shown in FIG. 11, step 460 may comprise: (a) transferring the step 450 mixture to dialysis membranes; and (b) removing unreacted glycidyl methacrylate from the mixture with the dialysis membranes. Dialysis steps 460 (a) and (b) may be performed at the target temperature maintaining step 410, such as 40° C.; and step 460 of method 400 may further comprise: (c) freezing the mixture to a target freezing temperature of −80° C.; and (d) lyophilizing the frozen mixture to obtain a dry product pH responsive polymer. Method 400 may comprise performing dialysis steps 460 (a) and (d) for a period of three days, during which freezing step 460 (c) may occur for a period between at least days and approximately seven days.

As shown in FIG. 7, the formation of Polymer B may be verified by 1H NMR. A decrease in the lysine peak at 3.00 ppm indicates the reaction of methacrylic anhydride with free amine groups. In addition, this is also indicated by the presence of signals displayed at 5.65 ppm and 5.42 ppm, which are the methacrylamide groups. The additional peak at 2.07 ppm observed for Polymer B is associated to the methyl carbon-linked hydrogens at the methacryl group, which also indicates the reaction with methacrylic anhydride. A small peak at 6.11 ppm indicates the reaction of methacrylic anhydride with hydroxyl(methacrylate) groups but represents one of the methacrylate vinyl protons because the other proton is overlapping with the peak at 5.65 ppm for methacrylamide. Thus, integration of the peak at 5.65 ppm represents the total amount of methacryl groups present in Polymer B.

### Benefits of Polymer A and B

When synthesized according to method 300 or 400, Polymer A and Polymer B may be used to enhance hydrogels, microgels, nanogels, and bioink compositions. In the case of hydrogels, adding pH responsive polymers containing acidic and basic pendants may accept or release protons according to the pH of the solution. Hydrogels containing carboxyl groups may become negatively charged at high pH, forming anionic polyelectrolytes. Amine groups, on the other hand, may become positively charged at low pH. The swelling behaviors of hydrogels with added pH responsive polymers may vary based on the presences of these functional groups and how they are modified, allowing certain pH responsive hydrogels to be utilized for specific applications. For hydrogels containing Polymer A, the presence of carboxyl, hydroxyl, and amino groups on the gelatin backbone allows for selective modification with methacryl groups, making it possible to realize swelling behaviors generally mirroring those of Polymer B.

Applications of pH responsive hydrogels are numerous, ranging from controlled drug carrier and delivery systems to sensors. For example, Polymer A hydrogels possessing basic functional groups like those described herein may be applied as drug delivery vehicles to cancerous environments. Growth of cancer cells within a healthy tissue culminates in remodeling the tissue environment, resulting in stiffening of the extracellular matrix and an increase of interstitial pressure. The latter, combined with the disorganized structure of the tumor area, may affect the clearance of waste products, leading to a decrease in pH in the tumor area as low as 5.6. In this example, Polymer A hydrogels may be used as drug delivery systems to release molecules at the tumor area by swelling when exposed to the acidic environment of the tumor area.

### Methods Applicable to Polymer A or B

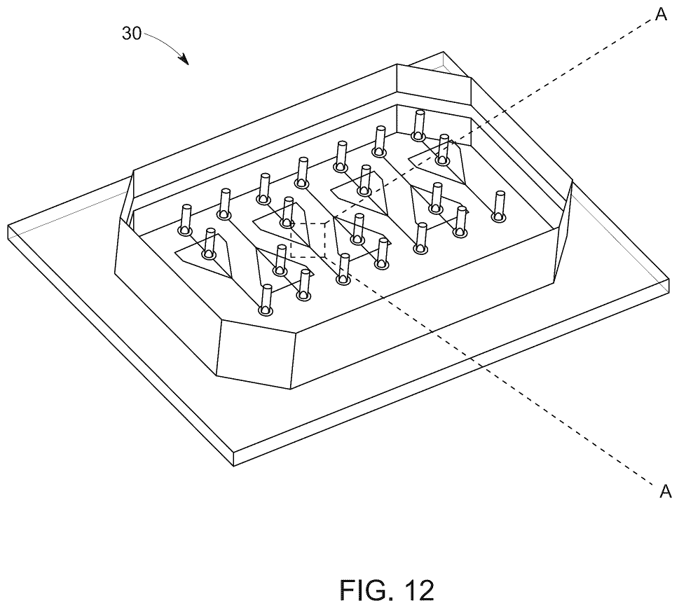

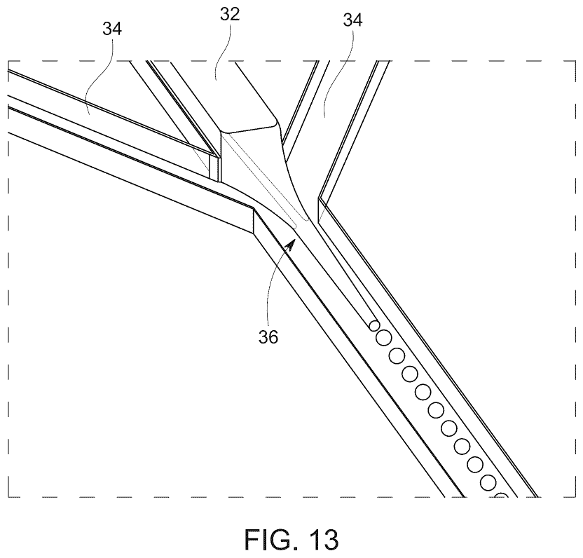

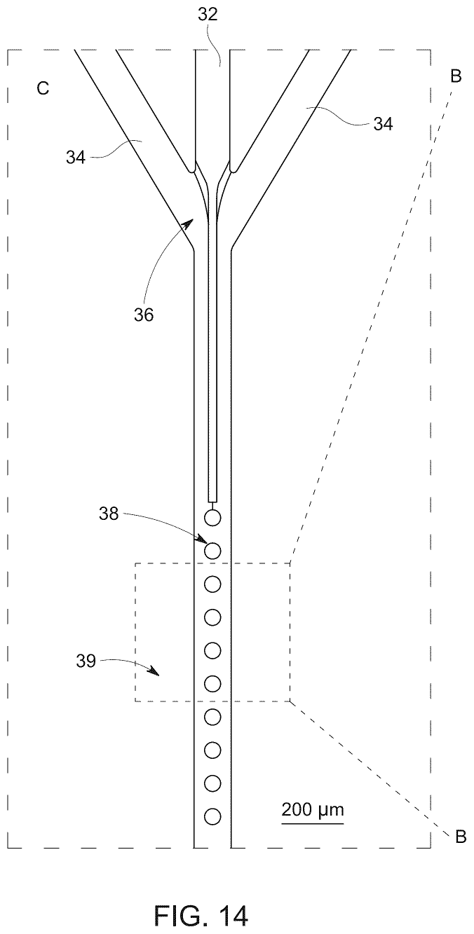

Methods 300 and 400 may comprise additional steps for processing pH responsive polymers, such as Polymers A and B. As shown in FIGS. 12 to 14, methods 300 and 400 may comprise steps for producing microspheres of Polymer A or Polymer B with a microfluidic device 30 having two phases of operation, including a continuous phase (e.g., within channels 32 and 34 described below and shown in FIGS. 13 and 14) and a dispersed phase (e.g., at cross-junction 36 described below and shown in FIGS. 13 and 14).

As shown in FIGS. 12, 13, and 14, microfluidic device 30 may comprise an inner channel 32 and outer channels 34. A solution comprising a pH responsive polymer and a photoinitiator may flow through inner channel 32 (e.g., from a pumping element) at a first flow rate. The pH responsive polymer may be Polymer A or Polymer B. Different types of photoinitiators may be used including one or more of irgacure 2959; Lithium phenyl-2,4,6-trimethylbenzoylphosphinate; sodium 3,3′-[(((1E,1′E)-(5-methyl-2-oxocyclohexane-1,3-diylidene)bis(methanylylidene)) bis(4,1phenylene))bis(methylazanediyl)]dipropanoate(E2CK); sodium3,3′-[(((1E,1′E)-(2-oxocyclopentane-1,3-diylidene)bis(methanylylidene))bis(4,1-phenylene))bis(methylazanediyl)]dipropanoate (P2CK); or tetrapotassium-4,4′-(1,2-ethenediyl)bis[2-(3-sulfophenyl)diazenesulfonate](AS7), another photoinitiator listed herein, and/or an equivalent thereof). For example, the solution may comprise 5% w/v of the pH responsive polymer (e.g., Polymer A or Polymer B) and 0.5% w/v of the photoinitiator (e.g., irgacure 2959, another photoinitiator listed herein, and/or an equivalent thereof) flowing through inner channel 32 at a first flow rate of 1 L/min. A temperature of inner channel 32 may be regulated to ensure the solution flows properly. For example, the temperature of inner channel 32 may be regulated at the operating temperature of maintaining steps 310 and 410, or approximately 40° C. A mixture of a mineral oil and a surfactant may flow through outer channels 34 at a second flow rate of 20 μL/min.

As shown in FIGS. 13 and 14, the solution in inner channel 32 and mixture in outer channels 34 may flow into a cross junction 36, creating a flow-focusing region, in which a stream of dispersed phase hydrogel microspheres 38 is developed because the surfactant in the mixture of surfactant and mineral oil stops the hydrogel microspheres from merging together. The first and second flow rates described above are exemplary as different sizes of hydrogel microspheres 38 may be created by adjusting the first flow rate of the solution flowing through inner channel 32 and/or the second flow rate of the mixture flowing through outer channels 34.

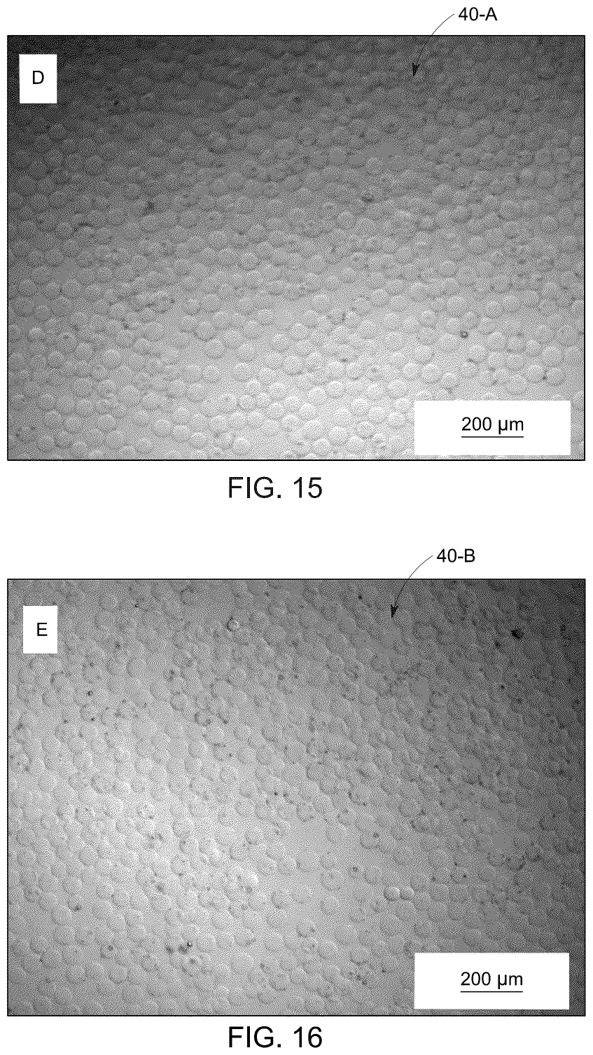

As shown in FIG. 14, hydrogel microspheres 38 may exit microfluidic device 30 and enter a meander tubing region 39 where they are selectively exposed to ultraviolet light for a crosslinking period of between approximately 0.5 and approximately 2 hours. The ultraviolet light may activate the photoinitiator mixed with Polymer A or Polymer B to polymerize the hydrogel microspheres into crosslinked hydrogel microspheres 40-A including Polymer A (e.g., FIG. 15) or crosslinked hydrogel microspheres 40-B including Polymer B (e.g., FIG. 16). Crosslinked hydrogel microspheres 40-A or 40-B may be collected and allowed to stabilize in the dark overnight and washed with Tetrahydrofuran (THF, 99.9%) or Hexanes to remove any excess amounts of the mineral oil, the surfactant, and/or the photoinitiator. Crosslinked hydrogel microspheres 40-A or 40-B may then be kept in a phosphate buffer solution for twenty-four hours for later use, such as within three days.

As shown in in FIGS. 15 and 16, respectfully, Polymer A and Polymer B hydrogel microspheres 40-A and 40-B may possess uniform size and shape, with diameters of 39.35±2.59 μm and 38.63±2.30 μm for particles produced from Polymers A and B. As noted above, different diameters may be realized by adjusting the first flow rate of the solution flowing through inner channel 32 and/or the second flow rate of the mixture flowing through outer channels 34.

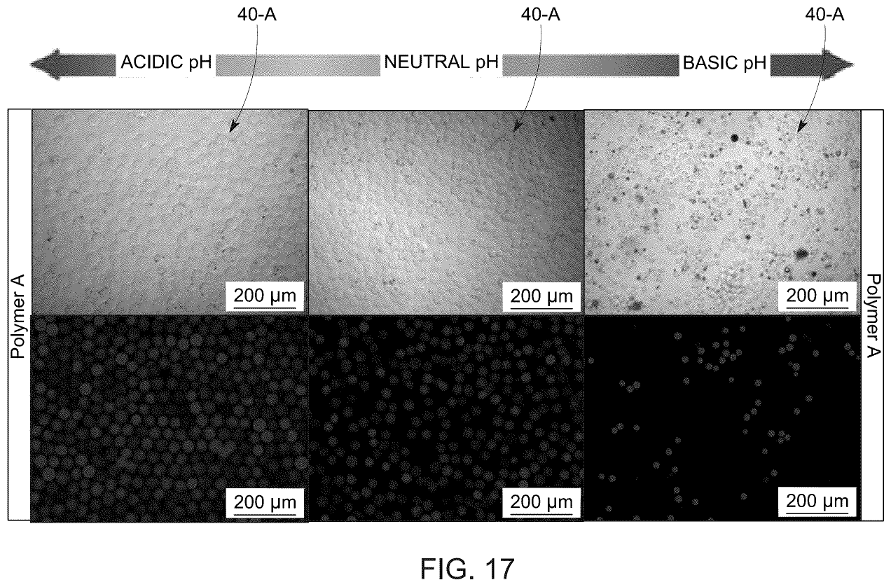

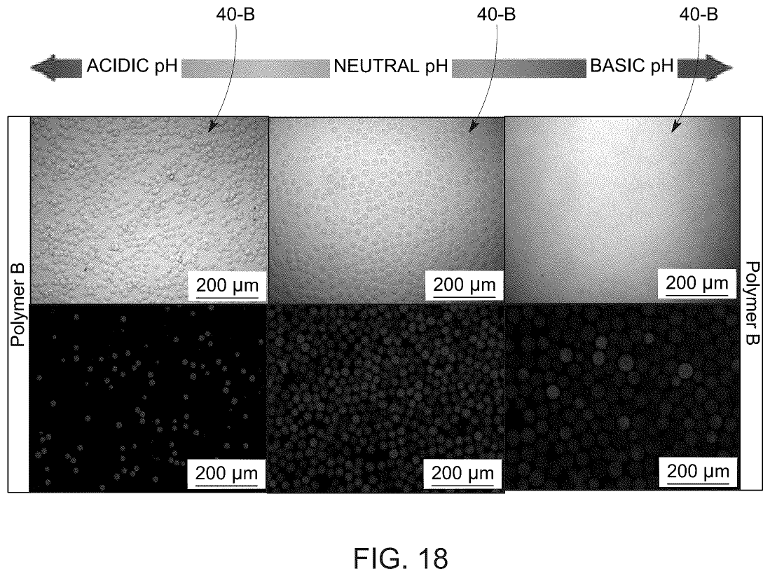

As shown in FIGS. 17 and 18, the swelling behavior of Polymer A and Polymer B hydrogel microspheres 40-A, 40-B may vary in acidic, neutral, and basic pH mediums. As shown in the bottom half of FIGS. 17 and 18, the swelling and shrinking of hydrogel microspheres 40-A, 40-B may be investigated by adding red-fluorescent polystyrene nanoparticles to the hydrogel microsphere solution to help tracking differences in the swelling behaviors.

FIG. 17 depicts swelling behaviors of Polymer A hydrogel microspheres 40-A in mediums with an acid pH (e.g., 6.0, at left), a neutral pH (e.g., 7.0, at middle), and a basic pH (e.g., 10.0, at right). As shown in FIG. 17, the swelling behaviors of Polymer A hydrogel microspheres 40-A may increase in diameter when exposed to the acidic pH medium and decrease in diameter when exposed to the basic pH medium. As shown in the bottom half of FIG. 17, the same swelling behaviors of Polymer A hydrogel microspheres 40-A may be displayed when red fluorescent polystyrene nanoparticles are added. In either instance, the swelling behaviors of Polymer A hydrogel microspheres 40-A may increase in diameter when in acidic mediums and decrease in diameter when in basic mediums. As shown in FIG. 17, the swelling and de-swelling of Polymer A hydrogel microspheres 40-A may display a swelling ratio, compared to when in neutral pH, of 166.6% in acidic pH (e.g., 6.0) and a de-swelling ratio of 61.3% in basic pH (e.g., 10.0).

FIG. 18 depicts swelling behaviors of Polymer B hydrogel microspheres 40-B in mediums with an acid pH (e.g., 6.0, at left), a neutral pH (e.g., 7.0, at middle), and a basic pH (e.g., 10.0, at right). As shown in FIG. 18, the swelling behaviors of Polymer B hydrogel microspheres 40-B may increase in diameter when exposed to the basic pH medium and decrease in diameter when exposed to the acidic pH medium. As shown in the bottom half of FIG. 18, the same swelling behaviors of Polymer B hydrogel microspheres 40-B may be displayed when red fluorescent polystyrene nanoparticles are added. In either instance, the swelling behaviors of Polymer B hydrogel microspheres 40-B may increase in diameter in basic mediums and decrease in diameter in acidic mediums. As shown in FIG. 18, the swelling and de-swelling Polymer B hydrogel microspheres 40-B may display a swelling ratio, compared to when in neutral pH, of 296.3% in basic pH (e.g., 10.0) and a de-swelling ratio of 57.6% in acidic pH (e.g., 6.0).

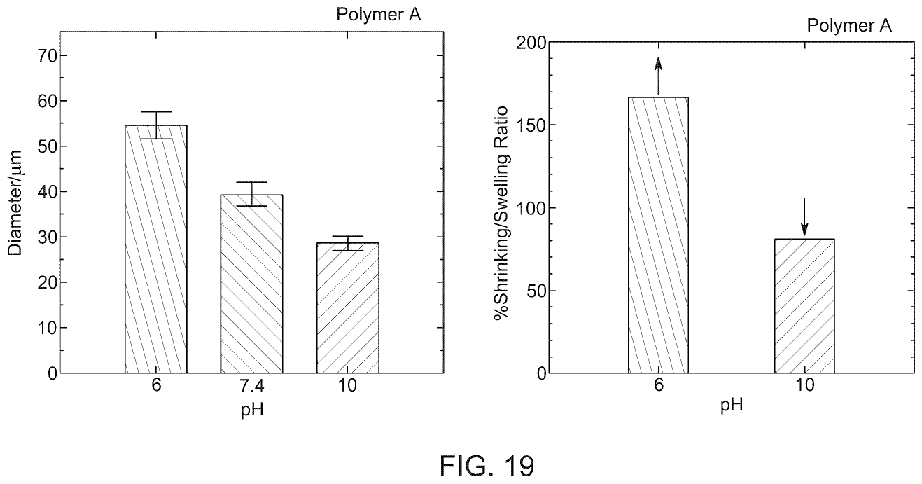

As shown in FIG. 19, aspects of Polymer A hydrogel microspheres 40-A may change according to a pH of the environment. As shown at left in FIG. 19, the diameter of Polymer A hydrogel microspheres 40-A may change from 39.35±2.59 μm at neutral pH of 7.0; to 54.56±3.03 μm at an acidic pH of 6.0; to 28.68±1.60 μm at basic pH of 10.0. As shown at right in FIG. 19, the swelling ratio of Polymer A hydrogel microspheres may comprise a swelling ratio of 166.6% in an acidic pH of 6.0; and a de-swelling ratio of 61.3% in a basic pH of 10.0.

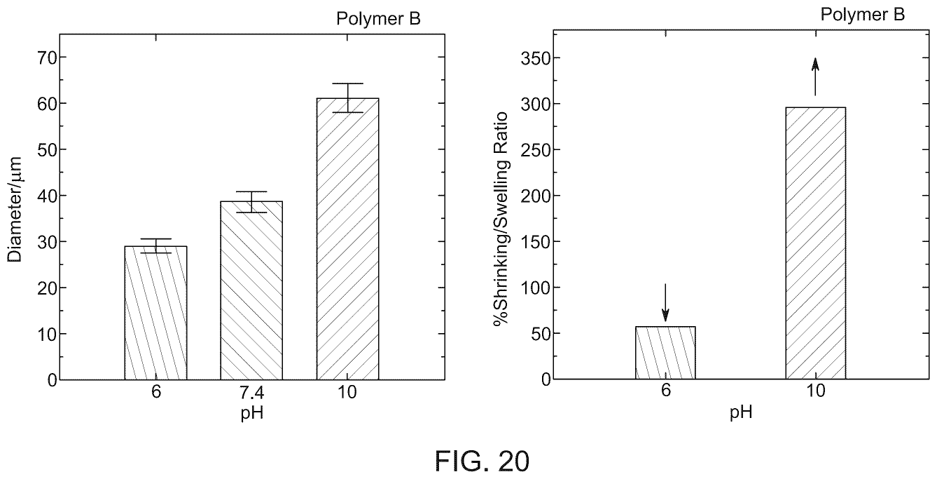

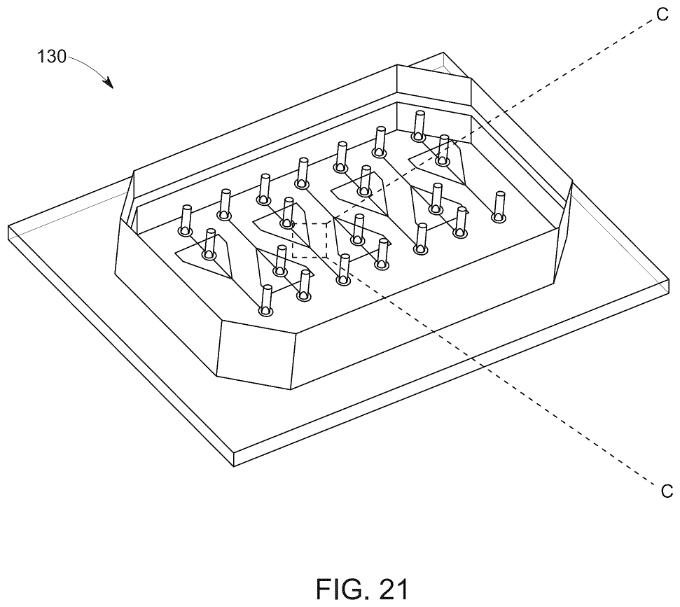

As shown in FIG. 20, aspects of Polymer B hydrogel microspheres 40-B may change according to a pH of the environment. As shown at left in FIG. 20, the diameter of Polymer B hydrogel microspheres 40-B may change from 39.35±2.59 μm at a neutral pH of 7.0; to 29.03±1.54 μm at an acidic pH of 6.0; to 61.13±3.22 μm at a basic pH of 10.0. As shown at right in FIG. 20, the swelling ratio of Polymer B hydrogel microspheres 40-B may comprise a swelling ratio of 296.3% in a basic pH of 10.0; and a de-swelling ratio of 57.6% in an acidic pH of 6.0.

When fabricated as described herein, Polymer A hydrogel microspheres 40-A and Polymer B hydrogel microspheres 40-B may contain acidic and basic pendants that may accept or release protons according to the pH of the solution. For hydrogels containing carboxyl groups, they may become negatively charged at high pH, forming anionic polyelectrolytes. Whereas, for hydrogels containing amine groups, they may become positively charged at low pH. As demonstrated in FIGS. 17 through 20, the swelling behaviors of Polymer A hydrogel microspheres 40-A and Polymer B hydrogel microspheres 40-B may be generally opposite from one another, in which Polymer A microspheres 40-A tend to swell in mediums where Polymer B hydrogel microspheres 40-B tend to shrink and vice versa.

In terms of drug delivery, the swelling behaviors of Polymers A and B may allow for specialized deployments of Polymer A hydrogel microspheres 40-A, Polymer B hydrogel microspheres 40-B, or combinations thereof based upon the pH of the drug or the treatment area. For example, Polymer A hydrogel microspheres 40-A may be applied as drug delivery vehicles to a cancerous environment with a low pH (e.g., as low as 5.6) causing microspheres 40-A to release molecules and/or particles by swelling when exposed to the cancerous environment. As a further example, Polymer B hydrogel microspheres 40-B may be similarly applied to biological environments with a high pH (e.g., higher than 7.4) causing microspheres 40-B to release molecules and/or particles by swelling when exposed to the biological environment.

Polymer A hydrogel microspheres 40-A and Polymer B hydrogel microspheres 40-B may be delivered together in a time released treatment. For example, Polymer A hydrogel microspheres 40-A and Polymer B hydrogel microspheres 40-B may be contained in a delivery medium that generally maintains the diameters of microspheres 40-A and 40-B, such as a mildly acidic water and/or mildly basic phosphate buffer solution. Polymer A hydrogel microspheres 40-A may contain a first treatment likely to increase a pH of a cancerous environment having an otherwise low pH (e.g., as low as 5.6) and Polymer B hydrogel microspheres 40-B may contain a related second treatment. Injecting the delivery medium to the cancerous environment may cause Polymer A hydrogel microspheres 40-A to release the first treatment by swelling when exposed to the cancerous environment, causing a pH of the cancerous environment to increase responsive to the first treatment. Polymer B hydrogel microspheres 40-B may then release the second treatment at some time after the first treatment was released, such as when a target pH the cancerous environment is realized.

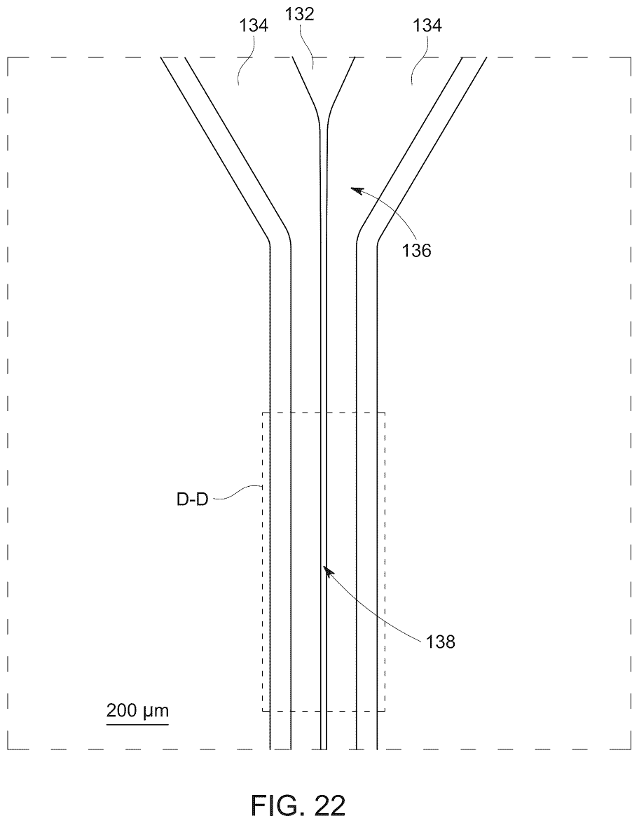

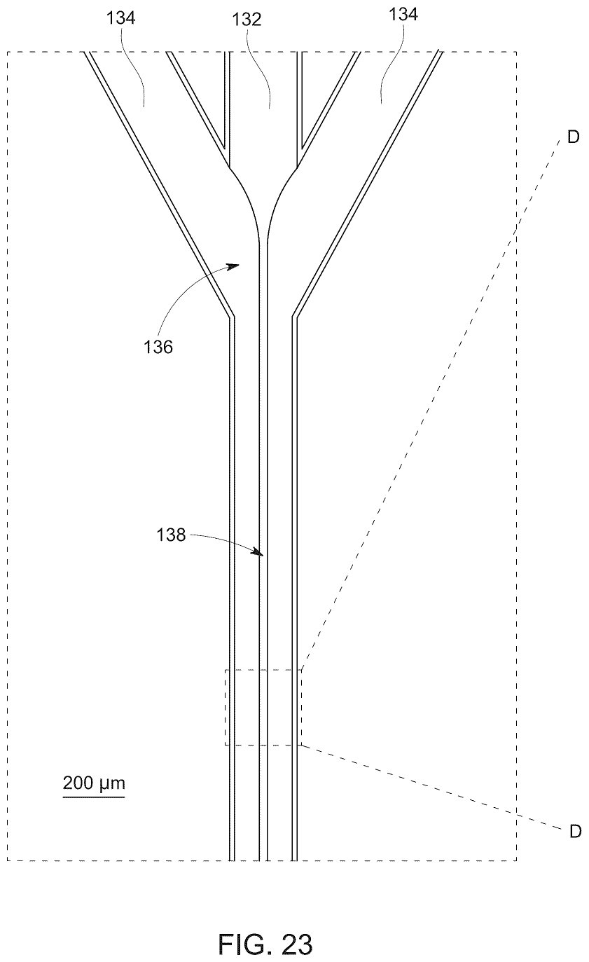

As shown in FIGS. 21 to 31, methods 300 and 400 also may comprise steps for producing nanospheres of Polymer A or Polymer B with a microfluidic device 130. As shown in FIGS. 22 and 23, microfluidic device 130 may have two phases of operation, including a solvent phase and a nonsolvent phase. As above, the nanospheres Polymer A or Polymer B may be similarly capable of encapsulating cargo for pH-dependent time release treatments.

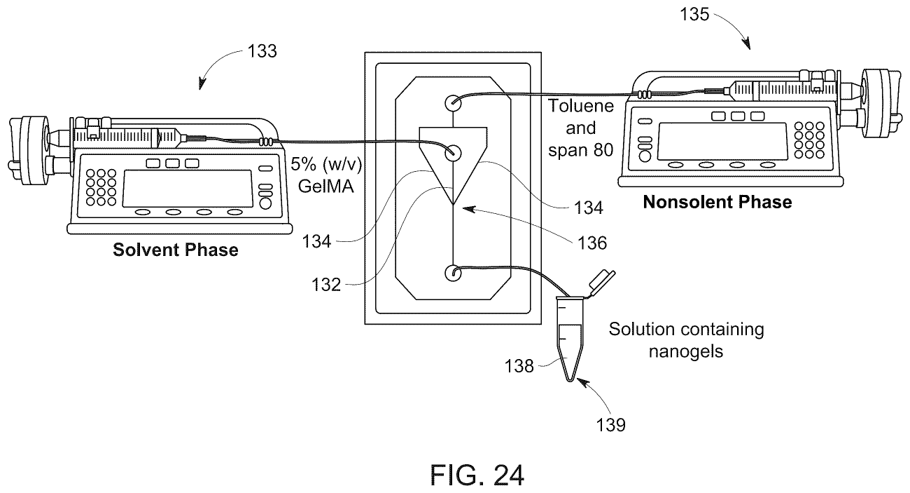

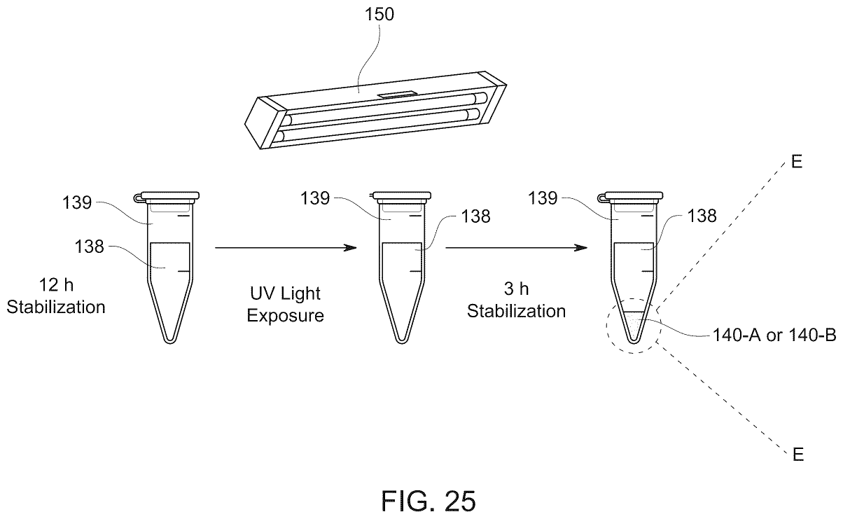

As shown in FIGS. 22 and 23, microfluidic device 130 may comprise an inner channel 132 and outer channels 134. As shown in FIG. 24, a solution comprising a pH responsive polymer and a photoinitiator may flow through inner channel 132 from a first pumping element 133 at a first flow rate. The pH responsive polymer may comprise Polymer A or Polymer B. Different types of photoinitiators may be used, including one or more of irgacure 2959; Lithium phenyl-2,4,6-trimethylbenzoylphosphinate; sodium 3,3′-[(((1E,1′E)-(5-methyl-2-oxocyclohexane-1,3-diylidene)bis(methanylylidene)) bis(4,1phenylene))bis(methylazanediyl)]dipropanoate(E2CK); sodium3,3′-[(((1E,1′E)-(2-oxocyclopentane-1,3-diylidene)bis(methanylylidene))bis(4,1-phenylene))bis(methylazanediyl)]dipropanoate (P2CK); and/or tetrapotassium-4,4′-(1,2-ethenediyl)bis[2-(3-sulfophenyl)diazenesulfonate](AS7). For example, the solution may comprise 5% w/v of the pH responsive polymer (e.g., Polymer A or Polymer B) and 1-0.5% w/v of the photoinitiator (e.g., Lithium phenyl-2,4,6-trimethylbenzoylphosphinate, another photoinitiator listed herein, and/or an equivalent thereof) flowing through inner channel 132 at a first flow rate of 4.5 μL/min. A temperature of inner channel 132 may be regulated to ensure the solution flows properly. For example, the temperature of inner channel 132 may be regulated by first pumping element 133 at the operating temperature of maintaining steps 310 and 410, or approximately 40° C. As shown in FIG. 24, a mixture of an organic solvent solution (e.g., toluene) and a surfactant (e.g., span 80™) may flow through outer channels 134 from a second pumping element 135 at a second flow rate of 400 μL/min.

As shown in FIGS. 22 and 23, the solution in inner channel 132 and the mixture in outer channels 134 may flow into a cross junction 136, creating a flow-focusing region, in which a stream of dispersed phase hydrogel nanospheres 138 may be developed because the surfactant in the mixture of organic solvent solution and surfactant stops the hydrogel nanospheres from merging together. The first and second flow rates described above are exemplary as different sizes of hydrogel nanospheres 138 may be created by adjusting the first flow rate of the solution flowing through inner channel 132 and/or the second flow rate of the mixture flowing through outer channels 134. As shown in FIG. 24, settings of first pumping element 133 and/or second pumping element 135 may be modified to adjust the respective first flow rate and/or second flow rate.

As shown in FIG. 24, hydrogel nanospheres 138 may exit microfluidic device 130 and be collected in a container 139 for a stabilization period of approximately twelve hours. Container 139 may have transparent or translucent sidewalls allowing ultraviolet light to pass therethrough. As shown in FIG. 25, after the stabilization period, hydrogel nanospheres 138 in container 139 may be selectively exposed to ultraviolet light from an ultraviolet light source 150 for a crosslinking period of between approximately 0.5 and approximately 2 hours. The ultraviolet light may activate the photoinitiator mixed with Polymer A or Polymer B to polymerize hydrogel nanospheres 138 into crosslinked hydrogel nanospheres 140-A including Polymer A or crosslinked hydrogel nanospheres 140-B including Polymer B. As shown in FIG. 25, crosslinked hydrogel nanospheres 140-A or 140-B in container 139 may then be allowed to stabilize in the dark for a stabilization period of between at least three hours and overnight, then washed with a solution of Tetrahydrofuran (THF, 99.9%) or Hexanes to remove any excess amounts of the organic solvent solution, the surfactant, and/or the photoinitiator. Crosslinked hydrogel nanospheres 140-A or 140-B may then be kept in the solution of THF or Hexanes for twenty-four hours for later use, such as within three days.

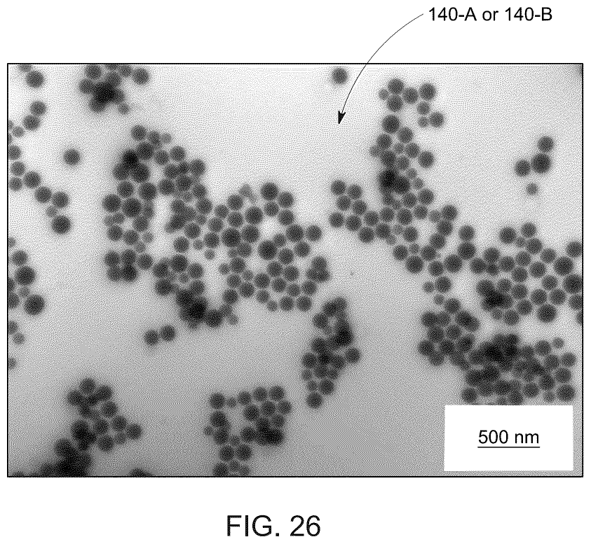

As shown in FIGS. 26, 27, and 29, Polymer A and Polymer B hydrogel nanospheres 140-A and 140-B may possess uniform size and shape, with diameters of 74±7 nm. As noted above, different diameters may be realized by adjusting the first flow rate of the solution flowing through inner channel 132 and/or the second flow rate of the mixture flowing through outer channel 134.

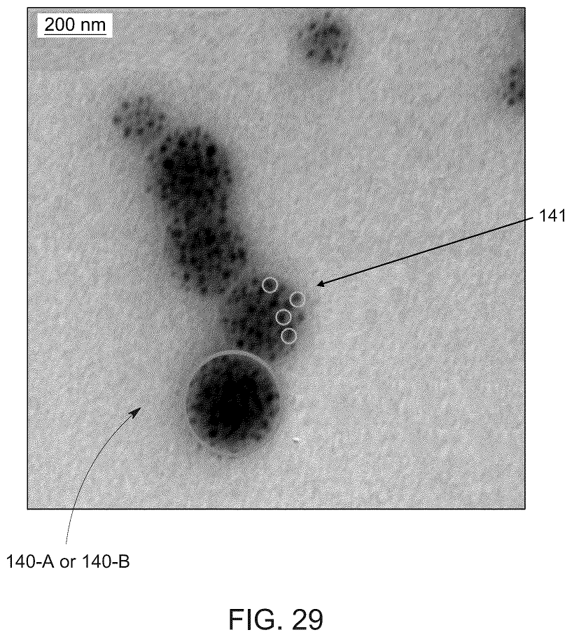

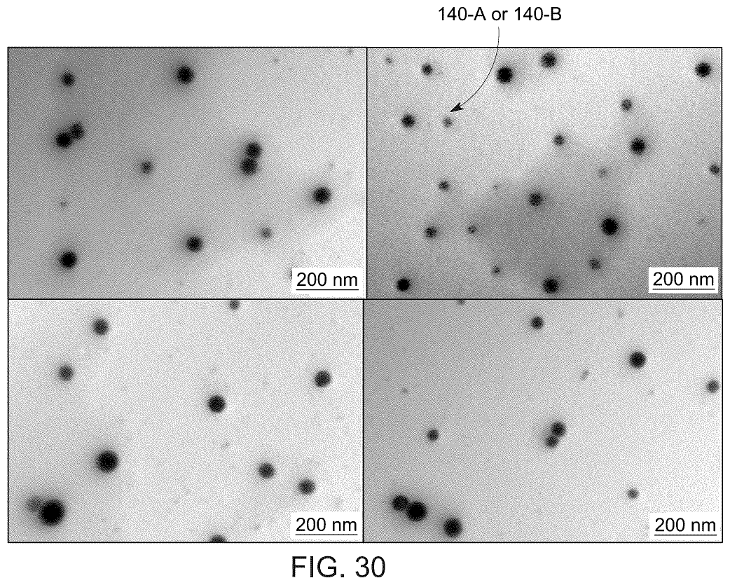

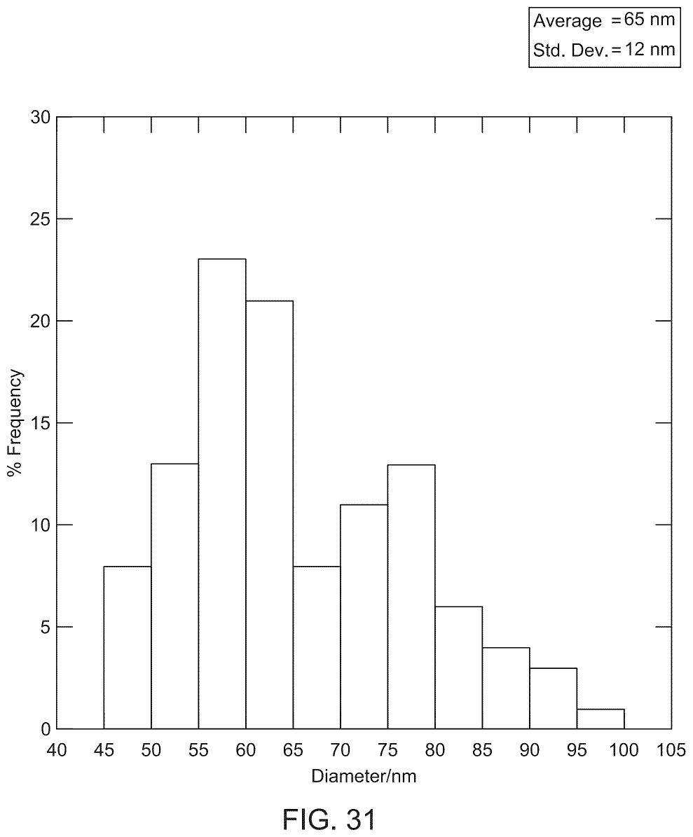

In keeping with FIGS. 21, 22, and 23, gold particles may be added to the solution flowing through inner channel 132 for encapsulation inside of crosslinked hydrogel nanospheres 140-A or 140-B. As shown in FIG. 29, gold nanoparticles 141 with a size of 7-10 nm may be added to the pH responsive polymer and/or the photoinitiator flowing through inner channel 132. Gold nanoparticles 141 may act as radiotherapy enhancers or sensitizers. As shown in FIGS. 30 and 31, crosslinked hydrogel nanospheres 140-A or 140-B containing gold nanoparticles 141 may possess uniform shape, with diameters of 66.27±13.24 nm, depending upon the first and second flow rates noted above.

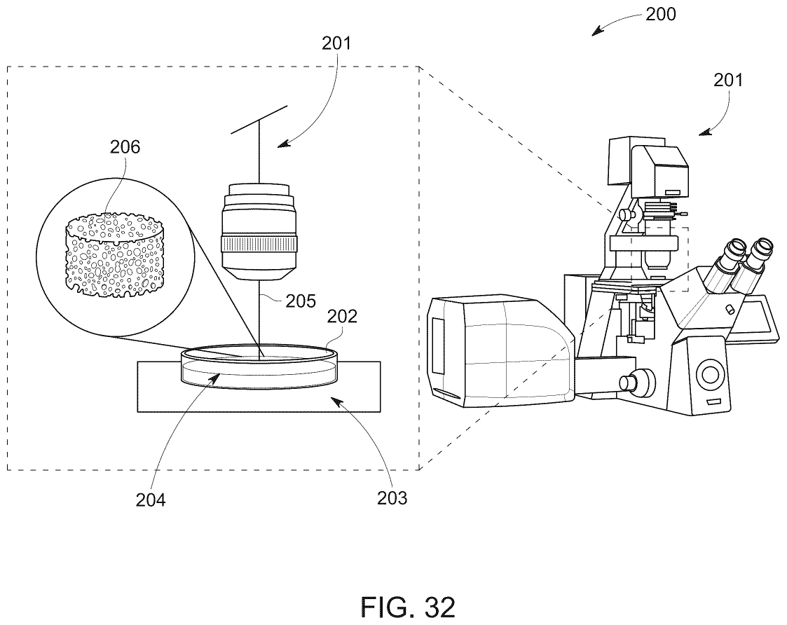

As shown in FIG. 32, methods 300 and 400 also may comprise steps for creating tissue constructs that are capable of mimicking artificial human tissue for the field of tissue engineering using 3D bioprinting technologies, such as lasers. Different combinations of pH responsive polymers (e.g., Polymer A or Polymer B) and photoinitiators (e.g., one or more of Lithium phenyl-2,4,6-trimethylbenzoylphosphinate; sodium 3,3′-[(((1E,1′E)-(5-methyl-2-oxocyclohexane-1,3-diylidene)bis(methanylylidene)) bis(4,1phenylene))bis(methylazanediyl)]dipropanoate(E2CK); sodium3,3′-[(((1E,1′E)-(2-oxocyclopentane-1,3-diylidene)bis(methanylylidene))bis(4,1-phenylene))bis(methylazanediyl)]dipropanoate (P2CK); and/or tetrapotassium-4,4′-(1,2-ethenediyl)bis[2-(3-sulfophenyl)diazenesulfonate](AS7)) may be utilized in tissue engineering applications. As shown in FIG. 32, a 3D bioprinting system 200 for tissue engineering may comprise a laser 201, a container 202, a controlled temperature plate 203, and a solution 204. Laser 201 may be operable to output a laser beam 205, such as 400 nm to 500 nm laser beam. Laser 201 may comprise a tunable laser (e.g., a tunable titanium:sapphire laser) so that laser beam 205 may be optimized for use with a particular photoinitiator. For example, laser beam 205 also may comprise a 300 nm laser optimized for use with tetrapotassium-4,4′-(1,2-ethenediyl)bis[2-(3-sulfophenyl)diazenesulfonate](AS7). Container 202 may comprise a petri dish. Controlled temperature plate 203 may be in the path of laser beam 205, used as a substrate for container 202, and be operable to regulate the temperature of solution 204 at approximately 2-4° C.

As shown in FIG. 32, solution 204 may comprise 10% w/v of the pH responsive polymer (e.g., Polymer A or Polymer B) and 0.045-0.1% w/v of the photoinitiator (e.g., one or more of Lithium phenyl-2,4,6-trimethylbenzoylphosphinate; sodium 3,3′-[(((1E,1′E)-(5-methyl-2-oxocyclohexane-1,3-diylidene)bis(methanylylidene)) bis(4,1phenylene))bis(methylazanediyl)]dipropanoate(E2CK); sodium3,3′-[(((1E,1′E)-(2-oxocyclopentane-1,3-diylidene)bis(methanylylidene))bis(4,1-phenylene))bis(methylazanediyl)]dipropanoate (P2CK); and/or tetrapotassium-4,4′-(1,2-ethenediyl)bis[2-(3-sulfophenyl)diazenesulfonate](AS7)). Solution 204 may be deposited in container 202 and placed onto controlled temperature plate 203. As shown in FIG. 32, laser beam 205 may be used to pattern a tissue construct 206 in solution 204. For example, tissue construct 206 may be patterned in solution 204 by crosslinking select portions of the pH responsive GelMA polymer with a laser beam 204 configured to the activate the photoinitiator at the select portions, such as a 300 to 500 nm laser beam tuned for use with the photoinitiator. Uncrosslinked portions of Polymer A and/or B in solution 204 may be removed by washing tissue construct 206 with a warm (e.g., approximately 25° C. or greater) flow of solution 204 and/or by utilizing controlled temperature plate 203 to increase the temperature of solution 204 to approximately 37° C., such that only tissue construct 206 remains in container 202. A plurality of different tissue constructs 206 may be patterned this way, providing a corresponding plurality of additional uses for Polymers A and B.

While principles of the present disclosure are described herein with reference to illustrative aspects, the disclosure is not limited thereto. Those having ordinary skill in the art and access to the teachings provided herein will recognize additional modifications, applications, aspects, and substitution of equivalents all fall in the scope of the aspects described herein. Accordingly, the present disclosure is not to be considered as limited by the foregoing description.

Embodiments in which an exclusive property or privilege is claimed are defined as follows:

1. A compound of formula (I) 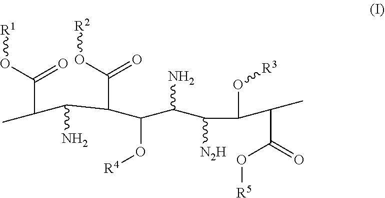
wherein:
the compound comprises a gelatin backbone;
R¹, R², R³, R⁴, and R⁵ are selected from a group consisting of: 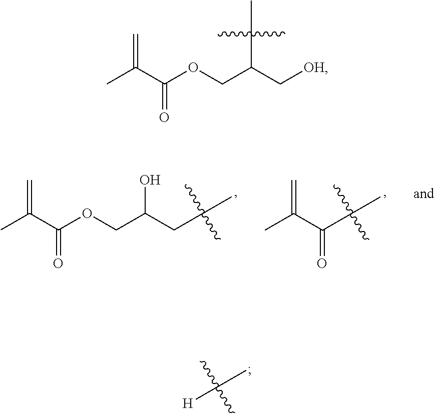
at least one of R¹, R², R³, R⁴, and R⁵ is not 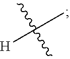 [SMILES: *[H]]
and “ [symbol] ” indicates the point of attachment.

2. The compound of claim 1, wherein

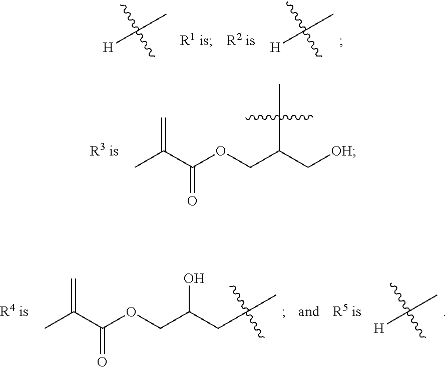

3. The compound of claim 1, wherein

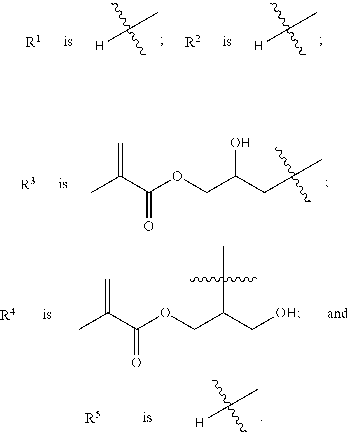

4. The compound of claim 1, wherein

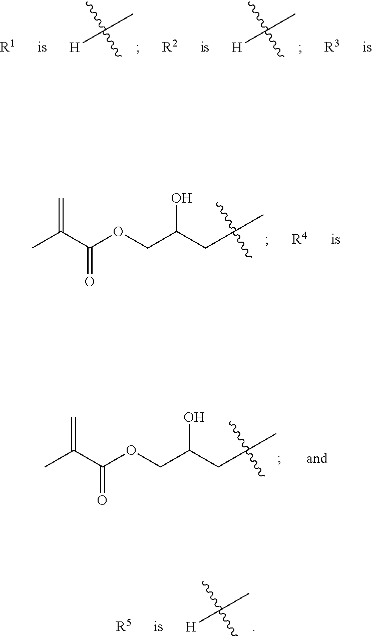

5. The compound of claim 1, wherein

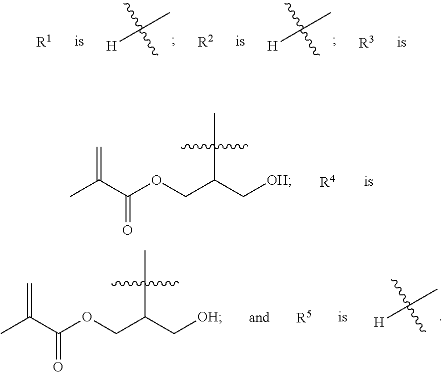

6. The compound of claim 1, wherein

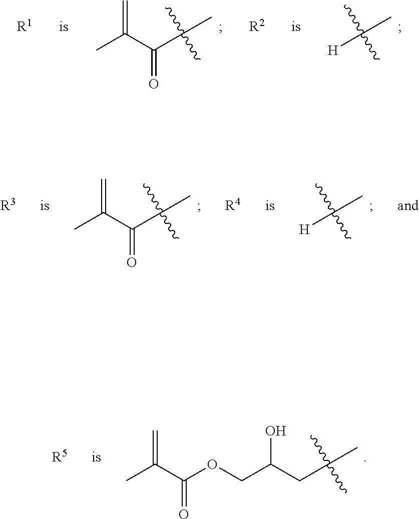

7. The compound of claim 1, wherein:

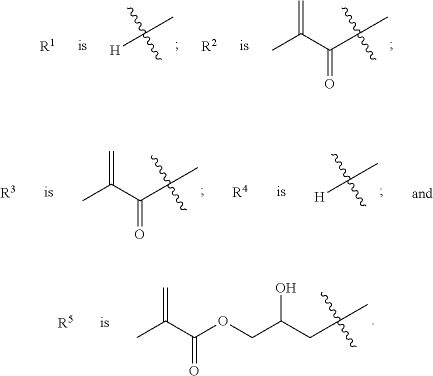

8. The compound of claim 1, wherein:

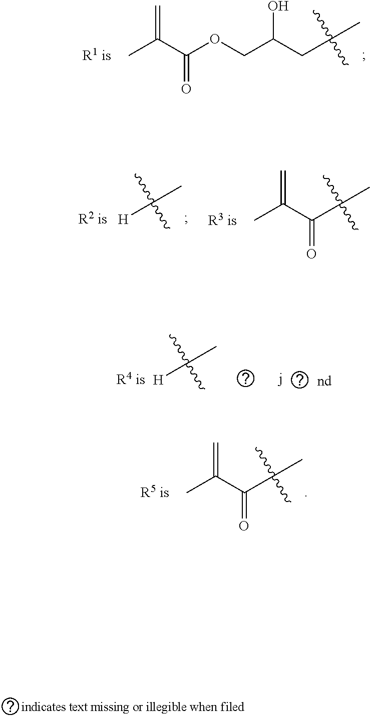

9. The compound of formula (I) according to claim 1, wherein:

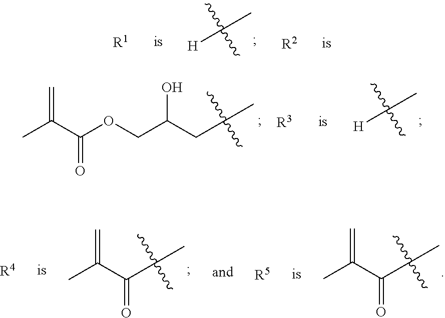

10. The compound of claim 1, wherein:

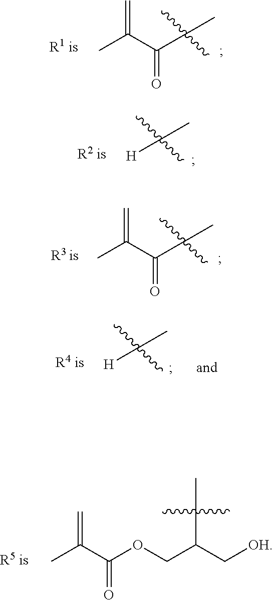

11. The compound of claim 1, wherein:

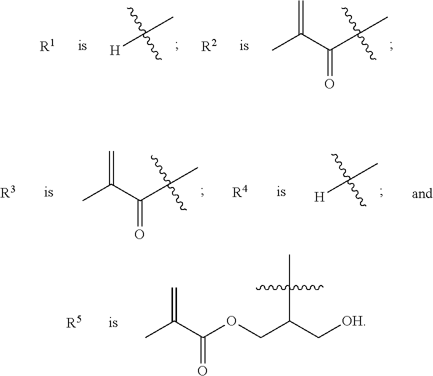

12. The compound of claim 1, wherein:

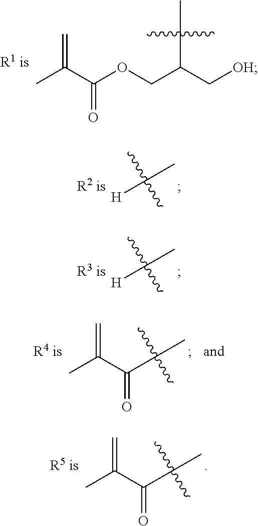

13. The compound of claim 1, wherein:

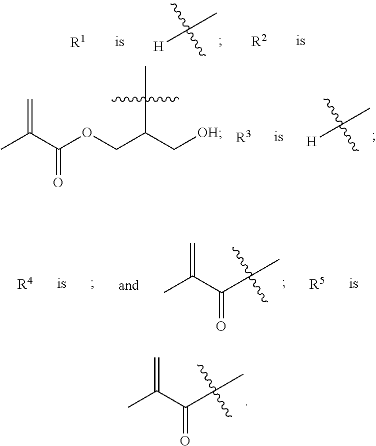

14. A method of synthesizing a pH responsive GelMA polymer comprising:
maintaining a vessel at a target temperature;
mixing a gelatin type A in an amount of an acidic water to form a solution in the vessel;
stirring the solution while:
beginning a reaction by adding glycidyl methacrylate to the solution thereby forming a mixture;
conducting the reaction for a first period while maintaining a target pH of the mixture by
measuring a pH of the mixture at intervals during the first period to determine a difference between the measured pH and the target pH, and
adding an additional amount of the acidic water to the mixture at each interval of the intervals of the first period so that the measured pH equals the target pH;
adding a second amount of the acidic water to the mixture after the first period;
conducting the reaction for a second period without maintaining the target pH;
adding a third amount of the acidic water to the mixture after the second period;
conducting the reaction for a third period; and
removing unreacted glycidyl methacrylate from the mixture after the third period.

15. The method of claim 14, wherein maintaining the vessel at the target temperature comprises placing the vessel in a temperature bath.

16. The method of claim 14, comprising maintaining the target temperature at approximately 40° C.

17. The method of claim 14, comprising maintaining the target temperature at between 40° C. and 50° C.

18. The method of claim 14, wherein the acidic water has a pH of 3.5.

19. The method of claim 14, comprising stirring the mixture at a rate of 400 rpm to 500 rpm.

20. The method of claim 14, comprising adding the glycidyl methacrylate to the solution dropwise at a flowrate.

21. The method of claim 20, wherein the flowrate is between approximately 0.1 mL per minute and approximately 0.5 mL per minute.

22. The method of claim 14, wherein the first period is approximately eighteen hours.

23. The method of claim 22, wherein the intervals during the first period occur approximately every five minutes for a first portion of the first period.

24. The method of claim 23, where the first portion of the first period is approximately three hours.

25. The method of claim 23, wherein the intervals during the first period occur approximately every thirty minutes for a second portion of the first period following the first portion of the first period.

26. The method of claim 25, wherein the second portion of the first period comprises a remainder of the first period.

27. The method of claim 14, wherein the intervals of the first period occur multiple times during each hour of the first period.

28. The method of claim 14, wherein the target pH of the mixture is 3.5.

29. The method of claim 22, wherein the second period is approximately six hours.

30. The method of claim 29, wherein the third period is approximately ten minutes.

31. The method of claim 14, wherein removing the unreacted glycidyl methacrylate from the mixture after the third period comprises:
dialyzing the mixture; and
lyophilizing the mixture.

32. The method of claim 31, wherein dialyzing the mixture comprises transferring the mixture to a dialysis membrane.

33. The method of claim 32, wherein dialyzing the mixture comprises performing dialysis, with the dialysis membrane, for a fourth period at a dialysis target temperature.

34. The method of claim 33, wherein the dialysis target temperature is 40° C.

35. The method of claim 33, wherein the dialysis target temperature is approximately 40° C.

36. The method of any one of claims 33 to 35, wherein the fourth period is approximately seven days.

37. The method of any one of claims 33 to 35, comprising adjusting the fourth period relative to a volume of the mixture being lyophilized.

38. The method of claim 33, wherein the fourth period is at least approximately days.

39. The method of claim 31, wherein the method comprises, prior to lyophilizing the mixture, exposing the mixture to a freezing temperature for a freezing period to create a frozen mixture.

40. The method of claim 39, wherein lyophilizing the mixture comprises placing the frozen mixture in a lyophilizer.

41. The method of claim 40, wherein lyophilizing the mixture comprises outputting, from the lyophilizer, a dry product of the pH responsive GelMA polymer.

42. The method of claim 39, wherein the freezing temperature is approximately negative eighty degrees Celsius (−80° C.).

43. The method of claim 39, wherein the freezing period is approximately seven days.

44. The method of claim 43, wherein the freezing period is at least three days.

45. The method of claim 39, comprising adjusting the freezing period relative to a volume of the mixture being lyophilized.

46. The method of claim 41, comprising synthesizing a hydrogel microsphere or a hydrogel nanosphere with the dry product.

47. The method of claim 46, comprising encapsulating molecules or particles in the hydrogel microsphere or the hydrogel nanosphere.

48. A pH responsive GelMA polymer comprising a gelatin type A with selectively modified and unmodified functional groups, the polymer comprising:
modified carboxyl groups, modified hydroxyl groups, unmodified carboxyl groups, unmodified hydroxyl groups, and unmodified amine groups;
carboxyl and hydroxyl groups that have been methacrylated; or
carboxyl, hydroxyl, and amine groups that were not methacrylated,
wherein the modified and unmodified functional groups of Polymer A may be selected by controlling a pH level of its synthesizing reaction, wherein the pH responsive GelMA polymer is synthesized according to the method of claim 14.

49. A pH responsive GelMA polymer comprising:
a gelatin type A comprising functional groups comprising
modified functional groups, and
unmodified functional groups,
wherein an acidic pH controls which functional groups are modified and unmodified.

50. The polymer of claim 49, wherein the modified functional groups consist of:
methacrylated carboxyl groups and methacrylated hydroxyl groups; or
methacrylated amine groups and methacrylated hydroxyl groups.

51. The polymer of claim 50, wherein the unmodified functional groups consist of:
unreacted amine groups;
unreacted carboxyl groups; and
unreacted hydroxyl groups.

52. The polymer of claim 51, wherein the acidic pH is 3.5.

53. A dry product comprising the polymer of any claims 49 to 52.

54. A solution comprising the dry product of claim 53 and a photoinitiator.

55. A Polymer B, namely, a pH responsive GelMA polymer comprising a gelatin type B with selectively modified amine groups, modified hydroxyl groups, unmodified carboxyl groups, unmodified, hydroxyl groups, and unmodified amine groups, wherein the pH responsive GelMA polymer is synthesized according to the method of claim 57.

56. The polymer of claim 55 comprising:
amine and hydroxyl groups that have been methacrylated; and
carboxyl, hydroxyl, and amine groups that were not methacrylated.

57. A method of synthesizing a Polymer B, namely, a photoinitiator pH responsive GelMA polymer, comprising:
maintaining a vessel at a target temperature of approximately 40° C.;
mixing a gelatin type B in an amount of a phosphate buffer to form a solution in the vessel;
conducting a reaction for a first period of approximately three hours by stirring the solution in the vessel while adding methacrylic anhydride to the solution thereby forming a mixture;
adding additional phosphate buffer solution to the mixture in the vessel and conducting the reaction for a second period of approximately six hours; and
removing unreacted methacrylic anhydride from the mixture after the second period by dialyzing the mixture; and
lyophilizing the mixture.

58. A method of synthesizing a photoinitiator GelMA polymer comprising:
forming a solution by combining a pH responsive GelMA polymer with a photoinitiator;
flowing the solution through an inner channel of a microfluidic device at a first flow rate while regulating a temperature of the inner channel;
flowing a mixture through outer channels of the microfluidic device at a second flow rate;
flowing the solution and the mixture into a cross junction of the inner channel and the outer channels, causing a plurality of hydrogel spheres to develop when the solution interacts with the mixture at the cross junction, each hydrogel sphere of the plurality of hydrogel spheres containing a separate volume of the solution;
exposing the plurality of hydrogel spheres to an ultraviolet light for a crosslinking period to activate the photoinitiator, creating a plurality of crosslinked hydrogel spheres; and
processing the plurality of crosslinked hydrogel spheres for later use.

59. The method of claim 58, wherein forming the solution comprises combining a dry product of the pH responsive GelMA polymer with the photoinitiator.

60. The method of claim 59, wherein the pH responsive GelMA polymer comprises Polymer A, namely a compound according to claim 1, or Polymer B, namely, a compound according to claim 55.

61. The method of any one of claims 58 to 60, wherein the photoinitiator comprises one or more of:
irgacure 2959;
Lithium phenyl-2,4,6-trimethylbenzoylphosphinate;
sodium 3,3′-[(((1E,1′E)-(5-methyl-2-oxocyclohexane-1,3-diylidene)bis(methanylylidene)) bis(4,1phenylene))bis(methylazanediyl)]dipropanoate (E2CK);
sodium3,3′-[(((1E,1′E)-(2-oxocyclopentane-1,3-diylidene)bis(methanylylidene))bis(4,1-phenylene))bis(methylazanediyl)]dipropanoate (P2CK); and
tetrapotassium-4,4′-(1,2-ethenediyl)bis[2-(3-sulfophenyl)diazenesulfonate](AS7).

62. The method of any one of claims 58 to 61, comprising modifying the generally uniform size of the hydrogel spheres by adjusting one or both of the first flow rate and the second flow rate.

63. The method of any one of claims 58 to 61, wherein regulating the temperature of the inner channel comprises maintaining the temperature of the inner channel at approximately 40° C.

64. The method of claim 63, wherein solution comprises:
5% w/v of the pH responsive GelMA polymer; and
0.5% of the photoinitiator.

65. The method of claim 64, wherein the first flow rate is 1 μL/min, the second flow rate is 20 μL/min, and causing a plurality of hydrogel spheres to develop comprises causing a plurality of hydrogel microspheres to develop, each microsphere of the plurality of microspheres having a generally uniform size.

66. The method of claim 65, wherein:
the mixture comprises a mineral oil and a surfactant; and
causing the plurality of hydrogel microspheres to develop comprises causing the surfactant in the mineral oil to stop the hydrogel microspheres from merging together at the cross junction of the microfluidic device.

67. The method of claim 65 or 66, wherein exposing the plurality of hydrogel microspheres to the ultraviolet light comprises:
directing the plurality of hydrogel microspheres into a tube; and
passing the ultraviolet light through an exterior wall of the tube for a crosslinking period of between approximately 0.5 and approximately 2 hours.

68. The method of claim 67, wherein processing the plurality of crosslinked hydrogel microspheres for later use comprises:
exposing the plurality of crosslinked hydrogel microspheres to a dark environment for an exposure time;
washing the plurality of crosslinked hydrogel microspheres with Tetrahydrofuran or Hexanes; and
storing the plurality of crosslinked hydrogel microspheres in a phosphate buffer solution for a storage time.

69. The method of claim 68, wherein the exposure time is overnight.

70. The method of claim 69, wherein the storage time is twenty-four hours.

71. The method of claim 63, wherein solution comprises:
5% w/v of the pH responsive GelMA polymer; and
between 1 and 0.5% of the photoinitiator.

72. The method of claim 71, wherein the first flow rate is 4.5 μL/min, the second flow rate is 400 μL/min, and causing a plurality of hydrogel spheres to develop comprises causing a plurality of hydrogel nanospheres to develop, each nanosphere of the plurality of nanospheres having a generally uniform size.

73. The method of claim 72, wherein:
the mixture comprises an organic solvent solution and a surfactant; and
causing the plurality of hydrogel nanospheres to develop comprises causing the surfactant in the mixture to stop the hydrogel nanospheres from merging together at the cross junction of the microfluidic device.

74. The method of claim 73, wherein the organic solvent solution comprises toluene.

75. The method of claim 74, wherein the surfactant comprises span 80™.

76. The method of any one of claims 72 to 75, comprising:
collecting the plurality of hydrogel nanospheres in a container; and
stabilizing the plurality of hydrogel nanospheres in the container for a stabilization period of approximately twelve hours.

77. The method of any one of claims 72 to 76, wherein exposing the plurality of hydrogel nanospheres to the ultraviolet light comprises:
directing the plurality of hydrogel nanospheres into a container; and
passing the ultraviolet light through an exterior wall of the container for a crosslinking period of between approximately 0.5 and approximately 2 hours.

78. The method of any one of claims 72 to 77, wherein processing the plurality of crosslinked hydrogel nanospheres for later use comprises:
exposing the plurality of crosslinked hydrogel nanospheres to a dark environment for an exposure time;
washing the plurality of crosslinked hydrogel nanospheres with a solution of Tetrahydrofuran or Hexanes; and
storing the plurality of crosslinked hydrogel nanospheres in the solution of Tetrahydrofuran or Hexanes for a storage time.

79. The method of claim 78, wherein the exposure time is at least three hours.

80. The method of claim 79, wherein the storage time is between twenty-four hours and thirty-six hours.

81. The method of claim 58, wherein the pH responsive GelMA polymer comprises Polymer A and the method comprises, after the processing step, one of:
exposing the plurality of crosslinked hydrogel spheres to an acidic environment that causes the plurality of crosslinked hydrogel spheres to expand; and
exposing the plurality of crosslinked hydrogel spheres to a basic environment that causes the plurality of crosslinked hydrogel spheres to shrink.

82. The method of claim 58, wherein:
the pH responsive GelMA polymer comprises Polymer A;
forming the solution comprises adding molecules or particles to the solution; and
causing the plurality of hydrogel spheres to develop comprises encapsulating the molecules or particles in the plurality of hydrogel spheres so that each hydrogel sphere of the plurality of hydrogel spheres contains a separate amount the molecules or particles.

83. The method of claim 82, comprising releasing the separate amounts of the molecules or particles by exposing the plurality of crosslinked hydrogel spheres to an acidic environment that causes the plurality of crosslinked hydrogel spheres to expand.

84. The method of claim 58, wherein the pH responsive GelMA polymer comprises Polymer B, namely, a compound according to claim 55, and the method comprises, after the processing step, one of:
exposing the plurality of crosslinked hydrogel spheres to an acidic environment that causes the plurality of crosslinked hydrogel spheres to shrink; and
exposing the plurality of crosslinked hydrogel spheres to a basic environment that causes the plurality of crosslinked hydrogel spheres to expand.

85. The method of claim 58, wherein:
the pH responsive GelMA polymer comprises Polymer B, namely, a compound according to claim 55;
forming the solution comprises adding molecules or particles to the solution; and
causing the plurality of hydrogel spheres to develop comprises encapsulating the molecules or particles in the plurality of hydrogel spheres so that each hydrogel sphere of the plurality of hydrogel spheres contains a separate amount the molecules or particles.

86. The method of claim 85, comprising releasing the separate amounts of the molecules or particles by exposing the plurality of crosslinked hydrogel spheres to a basic environment that causes the plurality of crosslinked hydrogel spheres to expand.

87. The method of any one of claims 81, 83, and 84, wherein the acidic environment comprises a cancerous environment.

88. The method of claim 82 or 85, comprising adding the molecules or particles to one or both of the pH responsive GelMA polymer and the photoinitiator.

89. The method of claim 82 or 85, wherein the molecules or particles comprise a radiotherapy enhancer or sensitizer.

90. The method of claim 82 or 85, wherein the molecules or particles comprise gold particles.

91. A method comprising:
producing a first plurality of crosslinked hydrogel spheres by
combining a first pH responsive GelMA polymer comprising Polymer A, namely a compound according to claim 1, with a first photoinitiator and first molecules or particles to form a first solution, and
forming the first plurality of crosslinked hydrogel spheres with the first solution according to method of claim 55;
producing a second plurality of crosslinked hydrogel spheres by
combining a second pH responsive GelMA polymer comprising Polymer B, namely, a compound according to claim 55, with a second photoinitiator and second molecules or particles to form a first solution, and
forming the second plurality of crosslinked hydrogel spheres with the second solution according to method of claim 55; and
forming a time-released mixture by combining the first plurality of crosslinked hydrogel spheres and the second plurality of crosslinked hydrogel spheres.

92. The method of claim 91, comprising:
exposing the time-released mixture to an environment having an initial pH that causes the first plurality of crosslinked hydrogel spheres to swell and burst, releasing the first molecules or particles at a first time;
allowing the released first molecules or particles to affect the initial pH at a second time after the first time; and
causing the second plurality of crosslinked hydrogel spheres to swell responsive to the affected initial pH until the second molecules or particles are released.

93. The method of claim 92, wherein the environment is a cancerous environment, the initial pH is an acidic pH, and the allowing step comprises converting the acidic pH into a basic pH.

94. The method of claim 93, comprising:
exposing the time-released mixture to an environment having an initial pH that causes the second plurality of crosslinked hydrogel spheres to swell and burst, releasing the second molecules or particles at a first time;
allowing the released second molecules or particles to affect the initial pH at a second time after the first time;
causing the first plurality of crosslinked hydrogel spheres to swell and burst, releasing the first molecules or particles at the second time.

95. The method of claim 94, wherein the environment is a biological environment, the initial pH is a basic pH, and the allowing step comprises converting the basic pH into an acidic pH.

96. The method of claim 93 or 95, wherein:
the acidic pH is 5.6; and
the basic pH is 7.4.

97. A method of synthesizing a patterned GelMA tissue construct comprising:
forming a solution comprising
10% w/v of a pH responsive GelMA polymer, and
between 0.045 and 0.1% w/v of a photoinitiator;
regulating a temperature of the solution;
patterning a tissue construct in the solution by crosslinking select portions of the pH responsive GelMA polymer with a laser beam configured to the activate the photoinitiator at the select portions; and
removing uncrosslinked portions of the pH responsive GelMA polymer from the container.

98. The method of claim 97, wherein the pH responsive GelMA polymer comprises Polymer A, namely a compound according to claim 1, or Polymer B, namely, a compound according to claim 55.

99. The method of claim 97, wherein regulating the temperature comprises:
depositing the solution in a container;
placing the container on a temperature-controlled plate; and
regulating the temperature with the temperature-controlled plate by maintaining the temperature of the solution at between approximately 2 and approximately 4° C.

100. The method of claim 97, wherein the laser beam is between 300 and 500 nm.

101. The method of claim 97, wherein the removing step comprises one or both of:
washing the tissue construct with a flow of the solution; and
increasing the temperature of the solution to approximately 37° C.
